(19) United States
(12) Patent Application Publication
(10) Pub. No.: US 2011/0300543 A1
(43) Pub. Date: Dec. 8, 2011

(54) METHODS FOR MAKING INDUCED PLURIPOTENT STEM CELLS FROM MESENCHYMAL STEM CELLS

(75) Inventor: Timothy C. Wang, New York, NY (US)

(73) Assignee: The Trustees of Columbia University in the City of New York, New York, NY (US)

(21) Appl. No.: 13/126,617

(22) PCT Filed: Nov. 2, 2009
(86) PCT No.: PCT/US09/62947

§ 371 Date: Aug. 23, 2011

Related U.S. Application Data
(60) Provisional application No. 61/110,183, filed on Oct. 31, 2008.

(51) Int. Cl. C12N 5/071; G01N 21/64; G01N 33/566; C12Q 1/04; C12Q 1/68

(52) U.S. Cl. 435/6.12; 435/377; 435/34; 435/366; 435/7.92; 435/7.1

(57) ABSTRACT

The invention is directed to methods for making iPS cells from Mesenchymal Stem Cells (MSCs). In certain aspects the methods comprise expression of Oct4 in MSCs, thereby converting the MSCs to iPS cells.

This application claims priority to Application Ser. No. 61/110183 filed Oct. 31, 2008 the content of which is hereby incorporated in its entirety.

This invention was made with government support under grant Nos. 5R01CA120979-04 awarded by NIH/NCI, 5R01 CA120979-02 awarded by NIH, R01 DK060694. The government has certain rights in the invention.

All patents, patent applications and non-patent references cited herein are hereby fully incorporated by reference in their entirety. The disclosure of these publications in their entirety is hereby incorporated by reference to more fully describe the state of the art as known to those skilled in the art as of the date of the invention described and claimed herein.

### BACKGROUND

Stem cells are believed to hold much promise in the treatment of chronic diseases such as Parkinson's, diabetes and spinal cord injuries. While embryonic stem (ES) cells have shown the greatest potential in the past, a new type of stem cell called induced pluripotent stem (iPS) cell, has recently been reported. These new iPS stem cells can be generated from almost any cell type and appear to be essentially equivalent to ES cells. The advantage of iPS cells is that one could create “patient-specific” stem cells from an individual for treatment of that patient, thus eliminating the need for immunosuppression in use of such iPS cells. However, the generation of iPS cells is technically challenging, and while reproducible, is far from routine. The creation of iPS cells by some of the currently practiced methods requires using viruses to introduce 4 genes, Oct3/4, Sox2, c-Myc, and KLF4, simultaneously into starting cells, for example mouse embryonic fibroblasts (MEFs), and only 1 in 10,000 cells in the end become an iPS cell.

This invention solves problems of making iPS by providing a different type of a starting cell—a stem cell called a mesenchymal stem cell or MSC, which is present in the bone marrow of adults. These cells show some similarities to ES cells but can give rise to some but not all tissues. The MSC cells of the invention express 3 of the 4 genes that are critical for iPS cells, but do not express one gene (Oct 3/4) that is most closely associated with embryonic development.

### SUMMARY OF THE INVENTION

The invention is directed to methods for making induced pluripotent stem (iPS) cells. In certain embodiments, the invention is directed to methods for making reprogrammed or converted cells which cells have higher potency capacity or level, compared to the starting cells, e.g., MSCs. In certain embodiments, the invention is directed to methods for making reprogrammed or converted cells which have at least certain characteristics of ES cells, compared to the starting cells, e.g., MSCs. The methods of invention contemplate expression of Oct4. In other embodiments the methods of the invention contemplate expression of Oct4 and Sox2 in the starting cells, such as MSCs. The expression of Oct3 or Sox2 can be at various levels. In certain embodiments the MSCs can be a population of isolated MSCs. In certain embodiments, the methods of the invention do not include steps of transfection or contacting of the MSCs with cMyc, KLF4, Nanog, Sox2, or any combination thereof.

In certain aspect the invention provides methods for making iPS cells. In one embodiment the invention provides a method for making induced Pluripotent Stem (iPS) cells from mesenchymal stem cells (MSCs) comprising:
a) transfecting MSCs, for example a population of isolated MSCs, to express Oct3/4; and
b) culturing the transfected MSCs under appropriate conditions, thereby converting, at least a subset of, the isolated MSCs into iPS cells, or converted cells with higher level of potency compared to the isolated MSCs, or converted cells which have at least some characteristics of ES cells, such as but not limited to morphology, growth/doubling time, gene expression profile, potency potential, or any combination thereof.

In another embodiment the invention provides a method for making iPS cells from mesenchymal stem cells comprising: expressing Oct3/4, or expressing Oct3/4 and Sox2 in isolated MSCs, for example a population of isolated MSCs, and culturing the MSCs under appropriate conditions, thereby converting at least a subset of the population of MSCs into iPS cells, or converted cells with higher level of potency compared to the isolated MSCs, or converted cells which have at least some characteristics of ES cells, such as but not limited to morphology, growth/doubling time, gene expression profile, potency potential, or any combination thereof.

In another embodiment the invention provides a method for making iPS cells from mesenchymal stem cells comprising: contacting or exposing MSCs, for example a population of isolated MSCs, with Oct3/4, or with Oct3/4 and Sox2, and culturing the MSCs under appropriate conditions, thereby converting at least a subset of the population of MSCs into iPS cells, or converted cells with higher level of potency compared to the isolated MSCs, or converted cells which have at least some characteristics of ES cells, such as but not limited to morphology, growth/doubling time, gene expression profile, potency potential, or any combination thereof.

In another embodiment the invention provides a method for making iPS cells from mesenchymal stem cells comprising:
a) transfecting isolated MSCs to express Oct3/4 and Sox2; and
b) culturing the transfected MSCs under appropriate conditions, thereby converting at least a subset of the population of MSCs into iPS cells, or converted cells with higher level of potency compared to the isolated MSCs, or converted cells which have at least some characteristics of ES cells, such as but not limited to morphology, growth/doubling time, gene expression profile, potency potential, or any combination thereof.

In certain embodiments, the methods optionally comprise a step carried out before the step of culturing the MSCs of identifying MSCs which have increased level of Oct3/4 expression, Sox2 expression, or both, and culturing the MSCs which have increased levels of Oct3/4 expression, Sox2 expression, or both.

In certain embodiments, the methods of the invention consist essentially of the steps of the methods described herein. In certain embodiments, the methods for making iPS cells from mesenchymal stem cells consist of the steps of the methods described herein. In certain embodiments, the methods of the invention, including the methods for making iPS cells from mesenchymal stem cells, do not comprise a step of transfecting, contacting or exposing MSCs to cMyc, KLF4, Sox2, Nanog, Lin 28, or any combination thereof. In certain embodiments, the methods of the invention including the methods for making iPS cells from mesenchymal stem cells, do not comprise a step of transfecting, contacting or exposing MSCs to cMyc, KLF4, or the combination thereof.

In certain embodiments, the MSCs are transfected with a plasmid vector or a viral vector. In certain embodiments, the MSCs are primate MSCs. In other embodiments, the MSC are human MSCs.

In certain embodiments, the MSCs of the inventive methods comprise certain subpopulations of MSCs. In certain embodiments, the MSCs of the inventive methods consist essentially of certain subpopulations of MSCs. In certain embodiments, the MSCs of the inventive methods comprise subpopulations of MSCs which express any of K19, KLF4, c-Myc, Sox2, Nanog, or any combination thereof. These subpopulations of MSCs can express any of K19, KLF4, c-Myc, Sox2, Nanog, or any combination thereof. These subpopulations of MSCs can be CD44+, SSEA1+ and are Lin(−), CD45(−). These subpopulations of MSCs can be CD44+ and are Lin(−), CD45(−). In certain embodiments, the inventive methods contemplate a population of isolated MSCs which consists essentially of certain subpopulations of MSCs which express any of K19, KLF4, c-Myc, Sox2, Nanog, or any combination thereof. In certain embodiments these subpopulation of MSCs do not express detectable levels of Oct3/4. In certain embodiments, the inventive methods contemplate a population of isolated MSCs wherein the isolated MSCs comprise, or consist essentially of, a subpopulation of MSCs which are CD44+, SSEA1+ and are Lin(−), CD45(−). In certain embodiments, the inventive methods contemplate a population of isolated MSCs, wherein the isolated MSCs comprise, or consist essentially of, a subpopulation of MSCs which are CD44+, and are Lin(−), CD45(−). In certain embodiments, the isolated MSCs comprise, or consist essentially of, subpopulations of MSCs which express higher levels of any of K19, KLF4, or any combination thereof compared to the rest of the MSCs in the population. In certain embodiments, the MSCs of the inventive methods comprise subpopulations of MSCs, wherein the isolated human mesenchymal stem cells (MSCs) are selected or isolated on the basis of expression of any of K19, KLF4, or combination thereof. In certain embodiments, the MSCs of the inventive methods comprise subpopulations of MSCs, wherein the isolated mesenchymal stem cells (MSCs) consist essentially of a population of MSCs with increased expression of any of K19, KLF4, or combination thereof.

In certain aspects, the invention provides a method for making subject specific iPS cells comprising:
a) isolating MSCs from a subject;
b) exposing the isolated MSCs to Oct3/4, or Oct3/4 and Sox2; and
c) culturing the MSCs of step (b) under appropriate conditions, thereby converting the MSCs or at least a subset of the MSCs into subject specific iPS cells.

In certain aspects, the invention provides an iPS cell, reprogrammed cells or converted cells obtained by any of the methods of the invention. In certain embodiments, the MSCs are obtained from a post-natal individual. In certain embodiments, the MSCs are obtained from the bone marrow of a subject, for example by any of the methods described herein. In certain embodiments, the MSCs are not obtained by differentiation in vitro from stem cells, by stimulation with differentiating factors, for example but not limited by bone marrow stromal cell. In certain embodiments, the methods of the invention convert at least a subset of the isolated MSCs into iPS cells. In certain embodiments, the method of the invention convert at least a subset of the isolated MSCs into converted cells with higher level of potency compared to the isolated MSCs. In certain embodiments, the method of the invention convert at least a subset of the isolated MSCs into converted cells which have at least some characteristics of ES cells, such as but not limited to morphology, growth/doubling time, gene expression profile, potency potential, or any combination thereof.

In certain aspects, the invention provides isolated MSCs which express K19, KLF4, c-Myc, Sox2, Nanog, or any combination thereof. In certain embodiments, the levels K19, KLF4, or any combination thereof are increased compared to a general population of isolated MSCs. In certain embodiments, the isolated MSCs do not express detectable levels of Oct3/4. In certain embodiments, the invention provides a sub-population of isolated MSCs which are CD44+ and SSEA1+, and are Lin(−) and CD45(−). In certain embodiments, the invention provides a sub-population of isolated MSCs which are CD44+, and are Lin(−) and CD45(−). In certain embodiments, the isolated MSCs of the invention are isolated from a subject, wherein the subject will be recipient of iPS cells, converted or reprogrammed cells produced from the isolated MSCs.

In certain aspects the invention provides a subset of adult bone marrow-derived MSCs that differ from iPS and ES cells primarily in the absence of Oct3/4 expression, and that iPS cells can therefore be generated from these MSC subsets by forced expression of Oct3/4. In some embodiments, iPS cells can be generated from these MSC subsets providing into these MSCs Oct3/4, or Oct3/4 and Sox2. In some embodiments, iPS cells can be generated from these MSC subsets by forced expression of Oct3/4, or Oct3/4 and Sox2. In certain embodiments, the invention provides expression of full length sequence, functional variants, fragments or functional equivalents of Oct3/4 or Sox2. The forced expression of Oct3/4 and Sox2 can be achieved by any known method in the art. Methods for introducing or expressing nucleic acids, transiently or stably, into mammalian cells are known in the art. In one embodiment the expression from a viral vector. In another aspect, the expression from a plasmid.

In certain aspects, the invention provides that the plasticity and mesenchymal differentiation ability of MSCs, including but not limited to K-19-MSCs, such as K19-EGFP MSCs, can be altered by overexpression of Oct 3/4. In another aspect, the invention provides that iPS cells be developed from MSCs, such as but not limited to K19-MSCs, with the forced expression of one or two additional genes, or a combination thereof, for example but not limited to Oct3/4 and Sox2. In other aspects, the invention provides iPS cells derived from bone marrow mesenchymal cells that endogenously expressed KLF4.

In certain aspects, the invention provides that bone marrow derived mesenchymal stem cells (MSCs) that express cytokeratin 19 (K19) to contribute to the gastric epithelium. Recent studies with Helicobacter-infected mice have shown that bone marrow-derived cells (BMDC) can repopulate the gastric epithelium and progress to cancer. However, it has not yet been established which cellular subset can potentially contribute to the epithelium. In certain embodiments, MSCs cultures were established from whole BM and expression of K19 was detected in a minority (1 of 13) of clones by RT-PCR and immunostaining In certain embodiments, the invention provides that high K19-expressing MSC clones (K19GFPMSC) were selected by transfection of a K19-EGFP vector and isolation of GFP-expressing colonies. In certain aspects, the invention provides that incubation of MSCs with gastric tissue extract markedly induced mRNA expression of gastric phenotypic markers, and was observed to a greater extent in K19GFPMSCs compared to parental MSCs and mock transfectants. Both K19GFPMSCs and GFP-labeled control MSCs (GFPMSCs) gave rise to gastric epithelial cells after injection into the murine stomach. In addition, after blastocyst injections, K19GFPMSCs gave rise to GFP-positive gastric epithelial cells in all 13 pups, while only 3 of 10 offspring showed GFP-positive gastric epithelial cells after injection of GFPMSCs. While K19 expression could not be detected in whole murine bone marrow, H. felis infection increased K19-expressing MSC CFU numbers in the circulation. In certain aspects, the invention provides that bone marrow-derived MSCs can contribute to the gastric epithelium. In certain embodiments, the K19 positive MSC fraction appears to be the relevant subset. In certain embodiments, this fraction is induced by chronic H. felis infection.

In certain aspects, the methods and cells of the invention are directed to use of human MSCs, and the relevant and corresponding genes and markers as described and used herein, for example but not limited to human Oct4, Sox2, Klf4, c-Myc, Nanog, Lin28, K19, and so forth. In certain embodiments, the methods of the invention require high level of Oct4.

### BRIEF DESCRIPTION OF THE FIGURES

FIG. 1 shows that MSCs can take on a gastric epithelial phenotype.

FIG. 2 shows that in MSCs K19+ Expression is Rare and Can Be Enriched in Clonal Culture.

FIG. 3 shows high K19-Expressing MSC Clones Selected by K19-EGFP-Vector.

FIG. 4 shows that K19-expressing MSCs show increased gastric differentiation ability.

FIG. 5 shows that MSC Can Give Rise to Gastric Epithelial Cells after blastocyst injection.

FIG. 6 shows expression of ES cell genes (Nanog, Sox2, c-Myc, KLF4, and Oct 3/4) by RT-PCR in K19GFPMSCs, parental MSCs and ES cells.

FIG. 7 shows KLF4-BAC-EGFP transgene. (Top) Diagram of the BAC transgene. (Bottom). GFP expression and KLF4 immunostaining in the small intestine.

FIG. 8 shows FACS for GFP expression in bone marrow from KLF4/EGFP transgenic mice.

FIG. 9 shows establishment of bone marrow derived MSC culture and induced expression of gastric phenotype markers after treatment with gastric tissue extract. FIG. 9A: Colony formation of MSCs. 500,000 or 1,000,000 cells of MSC at passage 5-10 were seeded onto 6 well tissue culture plate and colonies were visualized with crystal violet staining 14 days after plating. FIG. 9B: Adipocyte and osteocyte differentiation of MSCs. All established MSC cultures were incubated with adipocyte or osteocyte differentiation medium for 14 days and cells were stained with Oil red-O and Alizarin Red, respectively. FIG. 9C: Expression of cell surface markers (Sca1, c-kit, CD45, Flk1, and F4/80) was analyzed by flow cytometry. Quadrant markers were set according to the profile of corresponding control IgG staining Representative example of three experiments. FIG. 9D: Morphology of MSCs 5 days after treatment with gastric tissue extract. FIG. 9E: Expression of gastric epithelial phenotype markers in MSCs after treatment with gastric tissue extract. MSC was incubated with gastric tissue extract (GL) for 5 days and the mRNA expression of K19, TFF2, Muc5as, Muc6, H/K-ATPase, Intrinsic factor (IF), and chromogranin A (CGA) were detected by real-time PCR. Fold increase in mRNA expression (red bar) was showed, as compared to control cells which were incubated with culture medium without gastric tissue extract (blue bar) was calculated (n=3).

FIG. 10 shows identification of specific MSC clones which express the epithelial cytokeratin, K19. FIG. 10A: K19 expression was detected in MSC culture by immunofluorescence study with Texas Red conjugated anti K19 antibody. Nuclei were stained with DAPI. Original magnification, 100× (left panel). A high power view of a positive colony was set in right panel. FIG. 10B: Expression level of K19 mRNA in each MSC clone was quantified by real time PCR. Fold increase in each clone was showed, as compared to average expression level of all clones were shown. (n=3 in each clone) FIG. 10C: Alizarin Red and Oil red-O staining was performed to detect osteoblast and adipocyte differentiation, respectively, in 15 (No 1 to No 15) individual MSC clones isolated from the bone marrow of a single mouse, 14 days after treatment with appropriate differentiation medium.

FIG. 11 shows establishment of GFP-labeled MSC clones which express K19. FIG. 11A: GFP expression in MSC clones established by transfection with the K19-EGFP gene construct. After selection with G418 treatment, 3 of 11 isolated clones expressed GFP and were designated as K19GFP No 3, No 4, and No 5. FIG. 11B: Expression of GFP and cell surface markers (Sca1, c-kit, CD45, Flk1, and F4/80) in GFP positive clones were analyzed by flow cytometry. Cells were stained with PE conjugated antibodies, and both antibody staining and endogenous GFP expression was detected. Quadrant markers were set according to the profile of control IgG staining in GFP negative parent cells. Representative examples of three experiments are shown. FIG. 11C: Fold increase in K19 mRNA expression level in mock transfectant, K19GFP No 3, No 4, and No 5 was showed, as compared to that of parent cells by Real-Time PCR. (n=3). FIG. 11D: K19 protein expression in those GFP positive clones was assessed by immunofluorescent staining with Texas Red conjugated anti K19 antibody. Nuclei were stained with DAPI. Original magnification, 100×. FIG. 11E: Osteocyte and adipocyte differentiation 14 days after incubation with appropriate culture condition in K19GFP No 3, No 4, and No 5 were detected by Alizarin Red and Oil red-O staining, respectively. Original magnification, 40×.

FIG. 12 shows induced expression of gastric phenotype markers in K19 positive MSC clones after treatment with gastric tissue extract. K19GFP MSC clone No 3, No 4, and No 5 were incubated with gastric tissue extract for 5 days. FIG. 12A: The morphology of K19GFP MSC clones No 3, No 4, and No 5 after treatment with gastric tissue extract. Original magnification, 100×. FIG. 12B: Expression of gastric epithelial phenotype markers, such as k19, TFF2, Muc5ac, Muc6, H/K-ATPase, Intrinsic Factor (IF), and chromogranin A (CGA) in K19GFP MSC clones after treatment with gastric tissue extract (GL) were assessed by real time PCR. This was the average data of 3 separate experiments. FIG. 12C: Sorted GFP positive and negative cells were cultured separately and the expression of GFP was assessed 1, 7, and 28 days after culture. GFP negative cells could not generate GFP positive cells. Original magnification, 100×. FIG. 12D: Fold increase in K19 mRNA expression level in pooled K19GFP No 3, sorted GFP positive and negative fraction were compared to that of parent cells by real-time PCR. (n=3). FIG. 12E: The expression of gastric epithelial phenotype markers, such as K19, TFF2, Muc5ac, Muc6, H/K-ATPase, Intrinsic Factor (IF), and chromogranin A (CGA) in the sorted GFP positive and negative cells after treatment with gastric tissue extract (GL) were assessed by real-time PCR. FIG. 12F: BrdU assay showed higher proliferation ability in GFP (+) MSCs over GFP (−) MSCs (8 samples each, unpaired Student's t-test p=0.0029). FIG. 12G: GFP positive and negative cells were isolated by fluorescent cell sorting from K19GFP No 3. FIG. 12H: Colonies forming ability of single sorted GFP positive and negative cells. GFP positive cells could give rise to both GFP positive and negative cells, while GFP negative cells could not produce GFP positive cells. Original magnification, 100×. FIG. 12I: The expression of gastric epithelial phenotype markers, such as K19, TFF2, Muc5ac, Muc6, H/K-ATPase, Intrinsic Factor (IF), and chromogranin A (CGA) in the colonies derived from single sorted GFP positive and negative cells after treatment with gastric tissue extract (GL) were assessed by real-time PCR.

FIG. 13 shows contribution of K19-positive MSCs to the gastric epithelium in vivo. FIG. 13A: GFP MSCs (200,000 cells in 10 micro L of PBS) were injected into gastric wall of C57BL/6 mice and gastric tissue sections were prepared 2 weeks after injection. Sections were stained with anti E-cadherin antibody and Texas Red conjugated secondary antibody. Nuclei were stained with DAPI. Original magnification, 400×. FIG. 13B: K19GFP MSC No 4 (200,000 cells in 10 micro L of PBS) were injected into gastric wall of C57BL/6 mice and gastric tissue sections were prepared 2 weeks after injection. Sections were stained with anti E-cadherin antibody and Texas Red conjugated secondary antibody. Original magnification, 400×. FIG. 13C: GFP MSCs were injected into 3.5 day-old mouse blastocysts to establish chimeric mice and gastric tissue sections were prepared at 8-weeks of age. Sections were stained with anti E-cadherin antibody in combination with Texas Red conjugated secondary antibody. Nuclei were stained with DAPI. Original magnification, 400×. FIG. 13D: K19GFP MSC No 4 was injected into 3.5 day-old mouse blastocysts to establish chimeric mice and gastric tissue sections were prepared at 8 weeks of age. Sections were stained with anti E-cadherin antibody and Texas Red conjugated secondary antibody. Nuclei were stained with DAPI. Original magnification, 400×. FIG. 13E: Immunohistochemistry against GFP protein with anti GFP antibody. A representative result from stomach section of mouse derived from blastocyst injection of GFP MSC. Original magnification, 300×. FIG. 13F: Immuno fluorescent study for H/K-ATPase in GFP positive cells. Sections were stained with anti H/K-ATPase antibody and Texas Red conjugated secondary antibody. Nuclei were stained with DAPI. A representative result from stomach section of mouse derived from blastocyst injection of GFP MSC. Original magnification, 200×. FIG. 13G: GFP(+) cell detection rate and GFP(+) cells per high power field (HPF) in both gastric wall injection and blastocyst injection studies.

FIG. 14 shows K19-positive MSCs in the peripheral blood of mice with chronic H. felis infection. FIG. 14A: Expression of K19 mRNA in freshly isolated mononuclear cell fraction in bone marrow (BM) or peripheral blood (PB) of mice with or without H. felis associated chronic gastritis. FIG. 14B: Attached cell fraction 14 days after seeding of peripheral blood from uninfected control mice or mice with chronic H. felis infection. FIG. 14C: Adipocyte and osteocyte differentiation of MSC clones established from peripheral blood. PBMSCs were incubated with adipocyte or osteocyte differentiation medium for 14 days and cells were stained with Oil red-O or Alizarin Red, respectively. FIG. 14D: GFP positive cells in MSC culture established from peripheral blood of B6 mice which received bone marrow transplantation from chicken beta-actin EGFP donor mice and followed by chronic H. felis infection. FIG. 14E: Expression of gastric epithelial phenotype markers in peripheral blood derived MSC clones after treatment with gastric tissue extract (GL). Cells were incubated with gastric tissue extract for 5 days and the mRNA expression of K19, TFF2, Muc5as, Muc6, H/K-ATPase, intrinsic factor (IF), and chromogranin A (CGA) were detected by real-time PCR.

FIG. 15 shows expression of ES cell markers in cultured MSCs. Expression of ES cell markers, such as Nanog and Oct3/4, were investigated by RT-PCR. Relative expression level of Nanog mRNA was assessed by real-time PCR. Fold increase in mRNA expression was showed, as compared to ES cells.

FIG. 16 shows the comparison of gastric phenotypic gene expression in gastric tissue and K19GFP MSC with or without treatment of gastric tissue extract. The data were presented as cycle threshold. (n=3). FIG. 16A: Cytokeratin 19 (K19). FIG. 16B: MUC5AC. The level of MUC6 was similar to MUC5AC and omitted. FIG. 16C: Intrinsic factor (IF). FIG. 16D: Trefoil factor 2 (TFF2). FIG. 16E: HK-ATPase. FIG. 16F: Chromogranin A (CgA).

FIG. 17 shows that treatment of MSCs with gastric tissue extract, but not other tissue extracts, induces gastric phenotype marker expression. FIG. 17A: Morphology changes of MSC after treatment with colonic or pancreatic tissue extracts. FIG. 17B: Expression of gastric epithelial phenotype markers in parent MSC and K19GFP MSC No 4 5days after treatment with gastric, colon, or pancreas tissue extract was assessed by real-time PCR.

FIG. 18 shows establishment of MSCs from chicken beta actin EGFP transgenic mouse as a GFP labeled control cells. MSC culture was established from bone marrow of chicken beta actin EGFP transgenic mouse (GFP MSC). FIG. 18A: GFP expression in GFP MSC was assessed by fluorescent microscope and flow cytometry. FIG. 18B: Adipocyte and osteocyte differentiation of GFP MSC. MSC cultures were incubated with adipocyte or osteocyte differentiation medium for 14 days and cells were stained with Oil red-O and Alizarin Red, respectively. FIG. 18C: Expression of cell surface markers (Sca1, c-kit, CD45, Flk1, and F4/80) were analyzed by flow cytometry. Quadrant markers were set according to the profile of corresponding control IgG staining Representative example of three experiments.

FIG. 19 shows direct injection of MSCs into the murine stomach wall. Gastric tissue sections were prepared 24 hours after injection. FIG. 19A: GFP positive cells were detected in mucosa. FIG. 19B: GFP positive cells were detected in submucosal area. FIG. 19C: GFP positive cells were detected in subserosal area. Original magnification, 100× (upper panel). High power view was presented in lower panel.

FIG. 20 shows Blastocyst injection of GFP labeled MSC clones. FIG. 20A: GFP sequence was detected by PCR in tail DNA of mice derived from blastocyst injection of GFP MSC. FIG. 20B: GFP positive cells were detected in stomach tissue sections of 3 of 10 mice derived from blastocyst injection of GFP MSC. GFP positive cells were detected in subserosal area. FIG. 20C: GFP sequence was detected by PCR in tail DNA of mice derived from blastocyst injection of K19GFP MSC No 4. FIG. 20D: GFP positive cells were detected in stomach tissue sections of all 13 mice derived from blastocyst injection of K19GFP MSC No 4.

FIG. 21 shows co-localization of GFP and E-cadherin expression in gastric glandular cells detected by confocal microscopy. GFP positive cells detected under confocal microscope. No 4 stomach of mouse from blastocyst injection of GFP MSC (FIG. 21A, B), GFP MSC injection into gastric wall (FIG. 21C, D, E). FIG. 21A: GFP MSCs were injected into 3.5 day-old mouse blastocysts to establish chimeric mice and gastric tissue sections were prepared at 8 weeks of age. Four micrometer sections were stained with anti E-cadherin antibody in combination with Texas Red conjugated secondary antibody. Nuclei were stained with DAPI. FIG. 21B: 3D picture made from the same section in A. FIG. 21C: GFP MSCs (200,000 cells in 10 micro L of PBS) were injected into gastric wall of C57BL/6 mice and gastric tissue sections were prepared 2 weeks after injection. Four micrometer sections were stained with anti E-cadherin antibody and Texas Red conjugated secondary antibody. Original magnification, 400×. FIG. 21D: GFP single color photo. FIG. 21E: E-cadherin in Texas-red single color photo.

FIG. 22 shows Oct4 expression construct and a schematic representation of a method for converting MSCs to iPS cells.

### DETAILED DESCRIPTION

Converted or reprogrammed cells refer to cells which have increased potency compared to starting cells.

MSC: MSCs represent one subclass of bone marrow progenitors. They have also been called bone marrow-derived stem cells or BMDCs. In certain embodiments, MSCs are the bone marrow fraction that adheres to plastic petri dishes during culture. In certain aspects, the invention provides that Lineage (−), CD45(−) CD45(+) cells are the relevant MSC population that can be sorted from bone marrow.

Isolated MSCs which express cytokeratin 19 (K19) are referred to as K19-MSCs. In certain embodiments these include cells which express increased levels of K19 compared to the general population of MSCs. In certain embodiments, K19-MSCs are defined through transfection of K19-GFP vectors and selection of GFP+ cells.

Isolated MSCs which express KLF4 are referred to as KLF4-MSCs. In certain embodiments, these include cells which express increased levels of KLF4 compared to the general population of MSCs.

Until recently, the focus in the stem cell field centered on embryonic stem (ES) cells, cells derived from the inner cell mass of mammalian blastocysts that have the ability to grow indefinitely while maintaining pluripotency. Human ES cells are able to differentiate into cells of all three germ layers and might in theory be used to treat a number of chronic diseases—such as diabetes mellitus, Parkinson's disease and spinal cord injury—that are limited by the absence of significant tissue regeneration. Indeed, ES cells remain the standard for pluripotent cells, and no cell type has shown superior potential for plasticity and regeneration. However, the use of human ES cells, which are typically derived from human embryos, raised ethical concerns that have hindered the development and application of the technology.

A number of alternative approaches have been pursued to generate pluripotency in somatic cells, which have involved direct reprogramming by transferring their nuclear contents into oocytes or by fusion with ES cells (reviewed in Yamanaka 2007). However, in 2006, a radical new approach was reported by Takahashi et al [Takahashi 2006]. The group from Kyoto, Japan, reported that induced pluripotent stem (iPS) cells could be generated from mouse embryonic fibroblasts (MEFs) and adult mouse tail fibroblasts by the retrovirus mediated transfection of four transcription factors, Oct3/4, Sox2, c-Myc, and KLF4. The murine iPS cells were indistinguishable from ES cells in morphology, gene expression and teratoma formation, and give rise to chimeras and germline transmission when implanted into blastocysts [Takahashi 2006].

The work generated a great deal of excitement and has since been confirmed by several other groups [Wernig 2007; Yu 2007; Maherali 2007; Park 2008]. Both Yamanaka's group and others have demonstrated that the same approach can work to generate human iPS cells. One of the concerns regarding increased rates of tumorigenesis with iPS-derived tissues was lessened somewhat when it was shown that iPS cells could be made without c-Myc retroviral transduction [Nakagawa 2008]. The near-identity of iPS cells to ES cells was shown through epigenetic analysis, indicating that iPS cells have the same DNA methylation and gene expression patterns [Takahashi 2007a; Maherali 2007; Park 2008]. In addition, while it was speculated that all iPS cells might originate from stem or progenitor cells, work from several groups has pointed to the derivation of iPS cells from differentiated hepatocytes, gastric epithelial cells, pancreatic beta cells and lymphocytes [Aoi 2008; Stadtfeld 2008; Hanna 2008]. Other combinations of four transcription factors have been used [Yu 2007], but nearly all require both Oct 3/4 and Sox2. Finally, several studies have demonstrated the clinical utility of iPS cells in the treatment of rats with Parkinson's disease and mice with sickle cell disease [Wernig 2008; Hanna 2007].

While progress in the field has been extremely rapid the past two years, and published “protocols” have been made available to investigators [Takahashi 2007b], it remains unclear as to what cell type is best suited as the starting material for the generation of iPS cells. The introduction of 4 genes into somatic cells can be difficult. The use of c-Myc and retroviral vectors remain a concern; although lentiviral vectors can replace retroviral vectors in some systems, and Myc-less protocols can be used on embryonic fibroblasts (MEFs) [Nakagawa 2008], it is not clear that these approaches work well in adult cells. Most studies have used transgenic-based reporter genes (e.g. Fbx15-reporters or Nanog-reporters or Oct4-EGFP reporters) for selection, although selection of iPS cells by “morphology” has also been described [Meissner A 2007]. Typically, less than 10% of cells are infected with all four retroviruses, and less than 1% of the cells that have incorporated the four retroviruses can become iPS cells. Overall, the efficiency of the iPS protocol is in the range of 10-4. In theory, some of these obstacles could be overcome if iPS cells could be developed with transduction or transfection of only 1 or 2 genes, an approach that may be possible through greater selectivity in the cells used as starting material.

Induced pluripotent stem cells (iPS), which can be generated from adult somatic cells by forced expression of four (4) transcription factors, are emerging as a promising alternative to embryonic stem (ES) cells for potential use in regenerative therapy. However, iPS cells are technically very challenging to create and the overall process is of low efficiency. Very few groups have the skill and technical expertise to do this, and the low efficiency of the process remains a barrier to clinical and/or commercial use. In addition, the use of retroviruses and other viral vectors is a potential concern. To date, most iPS cells appear to have an increased risk of malignancy due to the presence of retroviral vectors and because of the introduction of c-Myc. While iPS cells have been generated from a variety of fetal and adult cell types, the ideal starting material for iPS development remains uncertain. The invention provides populations of isolated adult mesenchymal stem cells (MSCs), which possess significant plasticity and can cross germ layer boundaries, but are far less plastic than ES or iPS cells. The invention provides isolated types of MSCs (for example but not limited to K19-MSCs and pooled MSCs), characterized in in vitro culture studies and through blastocyst injection to be indeed multipotent and able to contribute to many tissues.

MSCs are easy to transfect. The invention provides methods of K19 screening or selection, including K19-EGFP selection, to identify specific subsets of MSCs that express KLF4, c-Myc, Sox2 and Nanog but not Oct3/4. The invention also provides use of KLF4 as a marker, for example KLF4-GFP transgenic mouse, to identify a subset of bone marrow mesenchymal cells that highly express KLF4, and these cells also express c-Myc and Sox2. Finally, the invention provides an endogenous subset of bone marrow MSCs that are Lin(−), CD45(−), CD44+ SSEA1+ that decrease with age and likely represent the primordial bone marrow stem cells. While they are rare in adults, they may also represent an ideal starting material for generation of iPS cells.

Given that MSCs are relatively easy to transfect using plasmid (nonviral) vectors, and that in theory only one gene may need to be introduced to create iPS cells, the invention provides methods for conversion of patient-specific MSCs to patient-specific iPS cells by expressing Oct4, or Oct4 and Sox2 by any method known in the art, for example using plasmid transfection of Oct 4, or Sox2. The invention provides methods to convert patient-specific MSCs, which can easily be isolated, for example but not limited to, by bone marrow biopsy, into iPS cells. In certain aspects, the methods include transfection of Oct4 into pooled MSC cells, K19-EGFP MSC cells, and KLF4-EGFP MSC cells, to convert these MSCs into iPS cells.

Induced pluripotent stem cells (iPS), which can be generated from adult somatic cells by forced expression of four transcription factors, are emerging as a promising alternative to embryonic stem (ES) cells for potential use in regenerative therapy. However, iPS cells are technically very challenging to create and the overall process is of low efficiency. In addition, the use of retroviruses and other viral vectors is a potential concern.

While iPS cells have been generated from a variety of fetal and adult cell types, the ideal starting material for iPS development remains uncertain. This invention provides adult mesenchymal stem cells (MSCs), which possess significant plasticity and can cross germ layer boundaries, but are less plastic than ES or iPS cells. MSCs are easy to transfect, and we have recently used K19-EGFP selection to identify a specific subset of MSCs that express KLF4, c-Myc, Sox2 and Nanog but not Oct3/4. We have also used a KLF4-GFP transgenic mouse to identify a subset of bone marrow mesenchymal cells that highly express KLF4, and these cells also express c-Myc and Sox2.

In certain aspect the invention provides methods to make iPS cells by introducing plasmid and/or lentiviral expression vectors for Oct 3/4 (with or without expression of Sox2) into K19-EGFP MSCs or parental MSCs. These methods can include examination of changes in gene expression and in vitro differentiation ability compared to untransfected controls. The combination of Oct4+Sox2 will be considered since MSC clones may express Sox2 at lower levels (20% of ES cell levels). K19-EGFP MSCs and parental MSCs that overexpress Oct3/4 (+/−Sox2) will be tested for pluripotency using a variety of in vivo models including blastocyts injections. In addition, we will examine perinatal death and test the tumorigenicity of chimeric mice derived from these blastocyst injections. Finally, we will continue our studies on KLF4 using KLF-EGFP transgenic mice that show KLF4 expression in a subset of bone marrow cells. We will isolate these cells and study their plasticity and gene expression patterns, followed by overexpression of Oct3/4 and/or Sox2 in order to test their ability to generate iPS cells. Methods described herein provide insight into the role of Oct3/4 in reprogramming MSCs, and generate iPS cells from these MSC clones. These methods simplify the process for generating iPS cells.

Bone marrow (MSC) stem cells are much closer to iPS stem cells, since they already express most of the genes needed except for one gene (Oct 4). Choosing the right starting cells will be critical for high yield production of iPS cells. Therefore, MSCs can be converted to iPS cells by overexpression of Oct4 in these cells. Expressing one missing gene (Oct3/4) into the adult mesenchymal stem cells provided herein is sufficient to increase the stem-like properties of these MSCs and can possibly change these stem cells into iPS cells, or cells that are equivalent to embryonic stem cells in being able to give rise to every tissue of the body.

General Methodology

Most research groups has not been selective about the cells used as starting material for generation of iPS cells, and consequently there has been a need to introduce four genes. The invention provides methods which use bone marrow stem cells, which show similarity to iPS cells, and provide different way to generate iPS cells.

The invention provides several lines of MSC cells that show an ability to give rise to multiple types of cells and tissues. Several different approaches can be used to introduce the one missing gene (Oct3/4) and then test these cells in tissue culture and in animal models for their ability to behave in a manner similar to iPS and ES cells. The methods of the invention will use bone marrow mesenchymal stem cells isolated from a subject that are carefully selected by the types of genes that they express. Some of them will likely be cultured for several weeks or more. The missing gene (Oct3/4) will be introduced into the cells by a process known as transfection or else using a special viral vector, and the MSCs then selected and tested for stem-like markers and properties. The cells will be tested and compared to iPS or ES cells based on their behavior when grown in Petri dishes or when injected into whole animals.

The methods of the instant invention provide significant advantages. MSCs can easily be obtained and grown from any patient, and it is much easier to introduce genes into these cells. The methods would allow rapid generation of patient-specific iPS cells. The studies will also provide new insight into the nature and potential of bone marrow stem cells.

The invention provides that bone marrow mesenchymal cells are not nearly as pluripotent as ES cells, yet express 3 of the 4 key genes needed for iPS formation. MSCs are inferior to ES cells in self-renewal and pluripotency, and do not express Oct4. The invention provides that forced expression of Oct4 in MSC subclones will convert the MSCs to cells that more closely approximate iPS cells. The main strategy here is to start with adult stem cells that are easily derived from any patient and thus still patient-specific, but that may be “closer” to iPS from the start and thus require fewer manipulations. These cells are more easily grown and manipulated than most adult cells. Thus forced expression of Oct4+/−Sox2 should increase the growth and plasticity of MSC cells and convert these cells to iPS cells. The invention also provides methods and populations of MSCs which used KLF4 as a marker of a subset of MSCs. The iPS-MSC cells of the invention can be tested in therapeutic models in mice. The methods of the invention contemplate using human MSCs. The invention also contemplates comprehensive epigenetic study of iPS cells.

Induced pluripotent stem (iPS) cells are undifferentiated cells similar to embryonic stem (ES) cells that have been generated from human and murine fibroblasts using retroviral transduction and forced expression of four transcription factors, but the process is technically challenging and of low efficiency. The invention provides a population of bone marrow progenitor cells with significant plasticity that already express certain markers. In certain embodiments, the invention provides a population of murine bone marrow progenitor cells with significant plasticity that already express c-Myc, KLF4, Sox2 and Nanog, but do not express Oct3/4. The invention provides human bone marrow progenitor cells with significant plasticity that already express c-Myc, KLF4, Sox2 and Nanog, but do not express Oct3/4.

In certain embodiments, these cells have been selected by transfection of mesenchymal stem cells with a K19-EGFP reporter gene and then selecting for high-expressing K19-EGFP(+) cells. K19-GFP(+) MSCs express higher levels of K19 transcripts and give rise to K19-GFP(−) MSCs, indicating that the former are progenitors. When injected into early-stage blastocysts, these K19-expressing cells show the ability to incorporate into the gastric epithelium and many other tissues, but do not express Oct3/4 and do not appear to be pluripotent. In addition, the invention provides isolated bone marrow mesenchymal cells expressing KLF4 at high levels, for example but not limited to cells from a KLF4-EGFP transgenic mouse. The invention provides methods for making iPS from select subpopulations of adult mesenchymal stem cells, for example but not limited to subpopulations of MSCS that express either K19 or KLF4 or both, following introduction of one gene (Oct4).

The invention provides that the plasticity and mesenchymal differentiation ability of K19-EGFP MSCs can be altered by overexpression of Oct 3/4. The invention provides methods to introduce expression vectors for Oct 3/4 into K19-EGFP MSCs or parental MSCs, and examine changes in gene expression and in vitro differentiation ability compared to untransfected controls.

The invention provides methods for making iPS from K19-MSCs by forced expression of (Oct3/4), or a combination of Oct3/4 and Sox2. In certain aspects K19-EGFP MSCs and parental MSCs that overexpress Oct3/4 can be tested for pluripotency using a variety of in vivo models including blastocyts injections. Perinatal death and test the tumorigenicity of chimeric mice derived from these blastocyst injections can be examined.

The invention provides methods for making iPS cells from bone marrow mesenchymal cells that endogenously express KLF4. The methods use KLF4-MSCs, for example KLF-EGFP transgenic mice, which show KLF4 expression in a subset of bone marrow cells. The invention provides methods isolate these cells and study their plasticity and gene expression patterns, followed by overexpression of Oct3/4 in order to test their ability to generate iPS cells.

In other aspects, the invention provides iPS cells derived from MSCs by any of the methods described herein.

Research on stem cells has great potential, both for developing new therapies for numerous intractable diseases and for advancing our understanding of basic human biology. While human embryonic stem (ES) cells continue to be an important research focus and should not be abandoned [Hyun 2007], the recent discovery of induced pluripotent stem (iPS) cells has broadened the possibilities for generating cells with properties closely matching those of ES cells. IPS cells have been generated from murine and adult fibroblasts and embryonic fibroblasts, as well as hepatocytes and gastric epithelial cells. While the generation of iPS cells by transduction of 3-4 genes is technically achievable, the invention provides methods for making iPS cells using as the starting material adult bone marrow stem cells, which can easily be isolated by marrow aspiration. The invention provides that the certain subsets of adult mesenchymal stem cells already possess significant plasticity and exhibit moderate levels of expression of key stem cell genes. In certain aspects, the invention provides iPS cells derived from MSCs.

A number of lines of evidence have pointed to possible plasticity of bone marrow-derived cells. Studies in gender-mismatched bone marrow transplantation recipients in human patients have provided evidence for the contribution of bone marrow derived cells (BMDCs) to epithelial cells of the liver [Thiese 2000], lung [Suratt 2003], and gastrointestinal tract [Okamoto 2002]. Bone marrow transplantation experiments in mice have similarly shown the engraftment of BMDCs in a variety of epithelial tissues, presumably assisting in the maintenance of the epithelium and speeding recovery from injury [Krause 2001]. MSCs constitutes only a small fraction of the bone marrow [Pittenger 1999] and originally were characterized as colony-forming fibroblast-like cells [Friedenstein 1974] that in culture adhered to plastic and possess the ability to differentiate into osteocytes, chondrocytes, adipocytes, and myocytes [Ferrari 1998]. Thereafter, numerous studies in humans and animals reported unexpected differentiation in vitro into endodermal tissues such as neural cells, cardiomyocytes, pneumocytes, and hepatocytes. More recently, a number of studies have demonstrated pluripotency of MSCs in vivo [Mackenzie 2001; Anjos-Afonso 2007; Jiang 2002]. The greatest plasticity has been observed for multipotent adult progenitor cells (MAPC) but isolation of these cells has been challenging and thus experience remains limited [Jiang 2002].

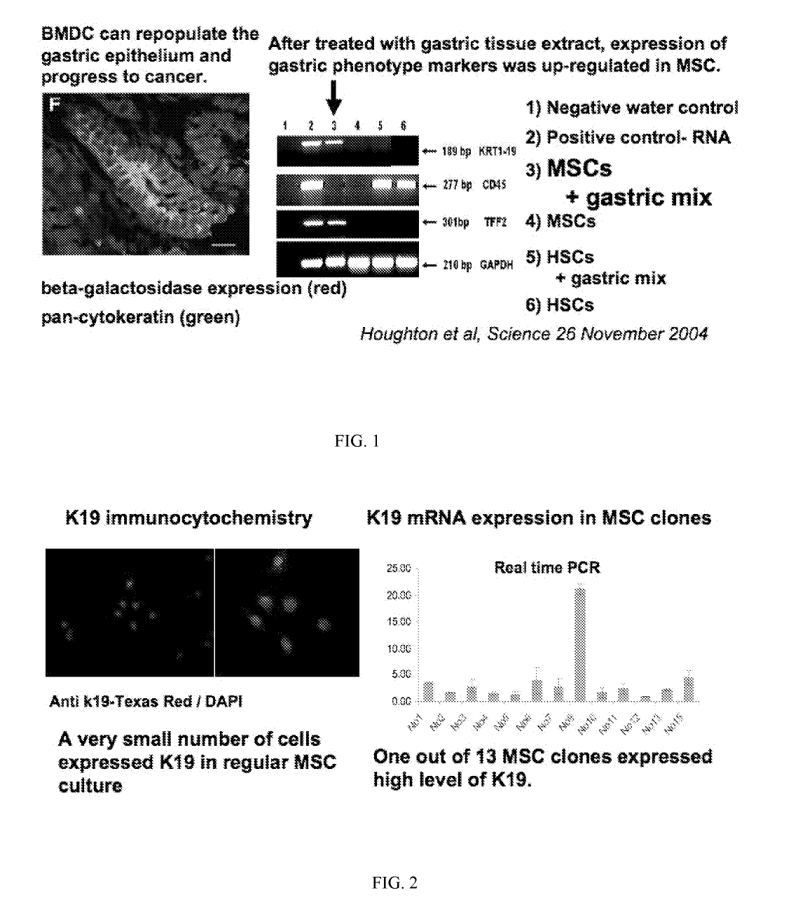

Our previous work provided the first clear evidence for the contribution of BMDCs directly to gastric epithelial metaplasia and dysplasia. In mice reconstituted with labeled bone marrow, chronic Helicobacter felis (H. felis) infection induced repopulation of the gastric epithelium with BMDCs, followed by progression of these cells to dysplasia (FIG. 1) [Houghton 2004]. In this earlier study, the mesenchymal stem cell (MSC) fraction of bone marrow was suggested as a possible source of BMDCs, since induction of gastric epithelial phenotype markers after treatment with gastric tissue extract was detected in the MSC fraction, but not in the HSC fraction [Houghton 2004]. Treatment with gastric extract of pooled MSCs resulted in a gastric-like morphology and increased expression of TFF2 and K19, two epithelial markers (FIG. 1).

In addition to their potential for differentiation into both mesodermal and endodermal lineages, some multipotent MSC subsets have been shown to express endoderm-associated genes, such as cytokeratins and Sox 17, along with known mesoderm-associated genes [Anjos-Afonso 2007; Nadri 2007; D'Ippolito 2004]. Cytokeratins are a family of intermediate filaments that are expressed in epithelial cells where they appear to be critical for the maintenance of the cytoskeleton [Moll 1982]. Among the various cytokeratins, keratin 19 (K19) has been reported to be expressed in the progenitor zone of the epidermis and the proliferative zone of the gastrointestinal tract, and is thought to be an early marker of epithelial cell lineage [Chun-mao 2007]. Importantly, K19 transgenes show expression in the gastric isthmus and bone marrow derived cells (BMDCs) were shown to consistently express K19 upon recruitment and engraftment in the stomachs of H. felis infected mice [Houghton 2004].

MSC cultures were established from whole bone marrow from mice as previously described based on their ability to adhere to plastic tissue culture dishes [Ferrari 1998]. Non-adherent cells were removed and the primary cultured MSCs became confluent within 2-3 weeks and grew exponentially for more than 15 passages without signs of senescence or differentiation. After 5 passages, the pooled MSCs demonstrated the abilities of colony formation and the ability to differentiate into both adipocyte and osteocyte lineages under previously defined conditions (not shown). Following treatment with gastric tissue extracts, the cultured MSCs altered their morphology from spindle-like fibroblastic to oblate or irregular appearance under phase contrast microscopy and showed increased expression of gastric epithelial phenotype markers such as K19, TFF2, MUC5AC, MUC6, H/K-ATPase, and chromogranin A. Flow cytometry (FACS) analysis of these primary MSC cultures revealed that majority of the cells expressed Sca-1 (94.4%), but not CD45, c-kit, or Flk-1.

The expression of K19 in the gastric progenitor zone and suggested that it might represent a good marker for early commitment to the gastric epithelial lineage. In addition, our data indicated that K19 was expressed at a low level or in a small subset of cultured MSCs. Immunofluorescence staining confirmed that a small number of cultured MSCs expressed K19 (FIG. 2). Individual MSC clones were isolated from primary cultures, expanded and then tested for K19 expression by RT-PCR. High levels of K19 mRNA expression could be detected in approximately one out of 13 clones (FIG. 2). This implied that, although K19 expression was present or could be included in a small subset of MSCs, K19+ cells could expand clonally and then be enriched for K19 expression. Most of these clones, including the clone with highest level of K19 expression, retained the ability to differentiate into osteocyte and adipocyte lineages under appropriate culture conditions.

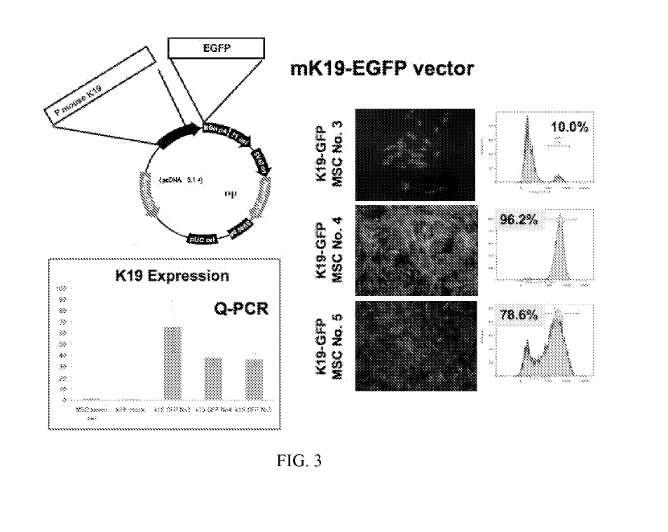

In order to isolate the minority of MSC clones that express K19, primary cultured MSCs were transfected with a K19-EGFP expression vector and stable clones were selected following G418 treatment. Fluorescence microscopy revealed 3 of 11 isolated clones to be positive for GFP expression (FIG. 3), and these clones (K19GFPMSC) were designated K19GFP No 3, No 4, and No 5. Flow cytometry revealed that the percentage of GFP positive cells was 10.0%, 96.2%, and 78.6% for K19GFP No 3, No 4, and No 5, respectively (FIG. 3). Real time PCR analysis showed 65-, 40-, and 35-fold increases in K19 mRNA expression level in K19GFP No 3, No 4, and No 5, respectively, compared to the parent MSCs (FIG. 3). Expression of K19 protein in the three stable MSC clones was confirmed by immunofluorescent staining FACS analysis revealed that expression of a variety of cell surface markers (including Sca-1, CD-45, c-kit, Flk-1, and F4/80) in the K19GFPMSC clones was roughly similar to that in the parent MSCs. All three K19GFPMSC clones retained the ability to differentiate into adipocyte and osteocyte lineages in culture.

GFP (+) cells were isolated by fluorescent cell sorting from the No 3 K19GFPMSC clone, in which only 10% of the cells expressed GFP, and confirmed enrichment of K19-expressing cells by Real-time PCR for K19. When the isolated GFP(+) and GFP(−) cellular fractions were cultured in MSC media for 28 days, GFP (+) cells gave rise to GFP (−) cells. In contrast, GFP (−) never gave rise to GFP (+) cells under the same conditions. These results suggest that the K19-expressing MSCs likely represent the progenitor cell fraction among pooled MSCs.

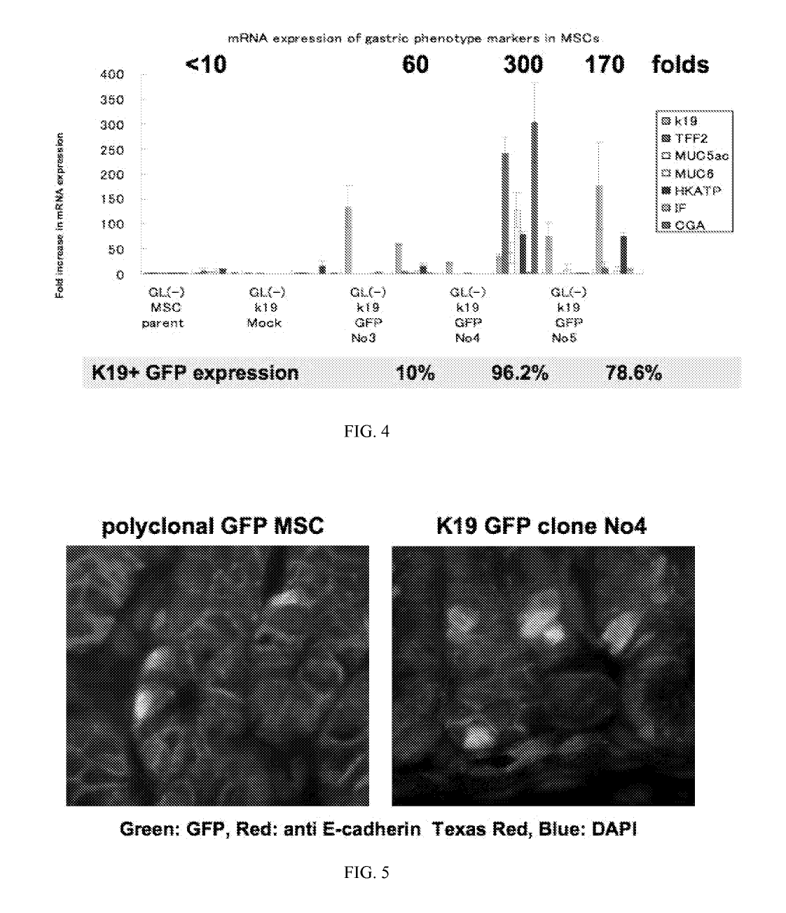

To evaluate the ability of K19 positive MSCs to differentiate toward gastric epithelial cells in vitro, the K19GFPMSC clones were treated with gastric tissue extract. Following five days of treatment, the cells altered their morphology from spindle-like to oblate or irregular shape, similar to what had been observed in the parental MSCs. In addition, the K19GFPMSC clones showed significant increases in mRNA expression of gastric phenotypic markers, with up to 60-, 300-, and 170-fold increases in K19GFPMSC clones No 3, No 4, and No 5, respectively (FIG. 4). In contrast, less than 10-fold increases were observed in the parental MSCs and mock transfectants (FIG. 4). This suggests that the potential of MSCs to express the gastric phenotype may be related to their level of K19 expression. The No 4 K19GFPMSC clone showed particularly high levels of gene expression of TFF2, MUC5AC, MUC6, H/K-ATPase, and chromogranin A, following treatment with gastric extract. In contrast, treatment of K19GFPMSCs clones with either colonic or pancreatic tissue extract did not induce upregulation of gastric phenotype markers in the K19GFPMSC clone No 4, but did upregulate colonic and/or pancreatic genes.

To evaluate the ability of K19 positive MSCs to differentiate toward a gastric epithelial cell lineage in vivo, the ability of various MSC clones to differentiate after injection into embryonic and adult mice was examined. The differentiation abilities of a representative clone, No 4 K19GFPMSC, was compared to GFP-labeled pooled primary MSCs. These latter MSC cultures were established from the bone marrow of chicken beta actin EGFP transgenic mice (designated control GFPMSC). The control GFPMSCs demonstrated a high degree (87.8%) of GFP expression, retained osteocyte and adipocyte differentiation, and expressed K19 and cell surface markers in a pattern similar to that of the parental MSC cultures derived from wild type C57BL/6 mice. To test the differentiation ability of these cells under embryonic conditions, we performed blastocyst injections of GFP-labeled MSCs. Following blastocyst injection of control GFPMSCs, GFP DNA sequence were detected by PCR in tail DNA from 6 of 10 chimeric offspring, and analysis of gastric sections from 8 week old mice showed GFP positive cells in the gastric epithelium in 3 of 10 animals. Some of the GFP+ gastric cells also showed E-cadherin positivity (FIG. 5). In comparison, GFP (+) cells could be detected in the gastric epithelium of all 13 pups derived from blastocyst injection of No 4 K19GFPMSCs with many GFP (+) E-cadherin (+) cells were distributed throughout the gastric epithelium (FIG. 5). The localization of GFP inside and E-cadherin on the membrane of the same cells was confirmed under confocal microscopy. Immunofluorescent study against GFP protein with anti GFP antibody further confirmed engraftment of injected MSCs into gastric mucosa, although expression of H/K-ATPase in GFP positive cells was not detected.

Overall, K19 GFP MSCs appeared to show longevity and more embryonic multipotentiality compared to parent MSCs, but they do not appear to be pluripotent, are clearly inferior to ES cells in this model, and could not give rise to germline transmission.

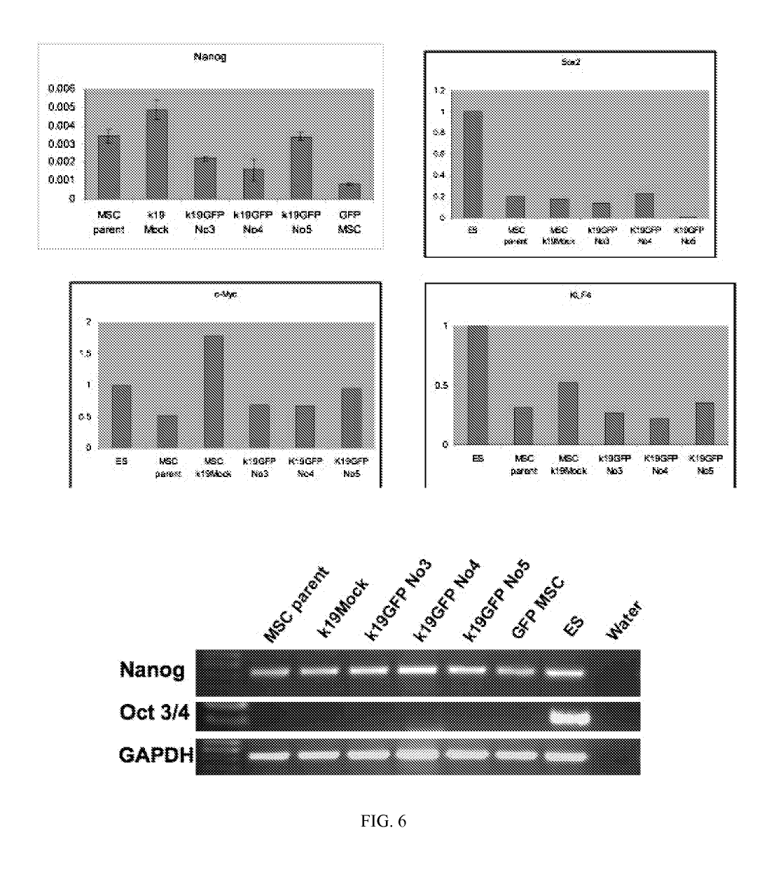

Nevertheless, K19GFP MSCs exhibited surprising plasticity. The expression of stem cell-related genes in these cells as well as in parent MSCs was examined. All three K19GFPMSC clones expressed Nanog, but not Oct3/4. However, they all expressed low levels of Sox2, and moderate levels—in the same range as ES cells—of KLF4 and c-Myc (FIG. 6). Overall, there was little difference in these genes between K19GFP MSCs and parent MSCs. Previous reports indicate that endogenous expression of c-Myc at a level of 20% of that of ES cell is sufficient for iPS generation [Nakagawa 2008]. Thus, these data lead to the interesting hypothesis that K19-GFP MSCs differ from ES cells primarily in their deficiency of Oct3/4 expression.

Of the four genes identified by Yamanaka as required for iPS induction, KLF4 is in some ways the most interesting and least understood [Yamanaka 2007]. KLF4 belongs to the family of Kruppel-like factors (KLFs), zinc-finger proteins that contain amino acid sequences resembling those of the Drosophilia embryonic pattern regulator Kruppel. KLF is highly expressed in differentiated, postmitotic epithelial cells of the skin and gastrointestinal tract. KLF4 is also expressed in fibroblasts lines such as NIH3T3 cells as well as embryonic fibroblasts (MEFs). Recent studies have also linked KLF4 to the Notch pathway. KLF4 is expressed at high levels in cells during growth arrest. It can function in both as a tumor suppressor and as an oncogene. Forced expression of KLF4 enables LIF-independent self-renewal and thus KLF4 likely plays a role in stem cell function.

KLF4 was isolated (previously known as gut-enriched Kruppel-like factor or GKLF) as one factor that binds to the gastrin responsive elements in the HDC promoter. KLF4 is a transcription factor that is highly expressed in the gastrointestinal tract. KLF4 bound to the gastrin responsive elements in the HDC promoter and overexpression of KLF4 dose dependently inhibited HDC promoter activity [Ai 2004]. We later showed that KLF4 interacts with a co-repressor, Tip60 and HDAC7 to mediate transcriptional repression [Ai 2007].

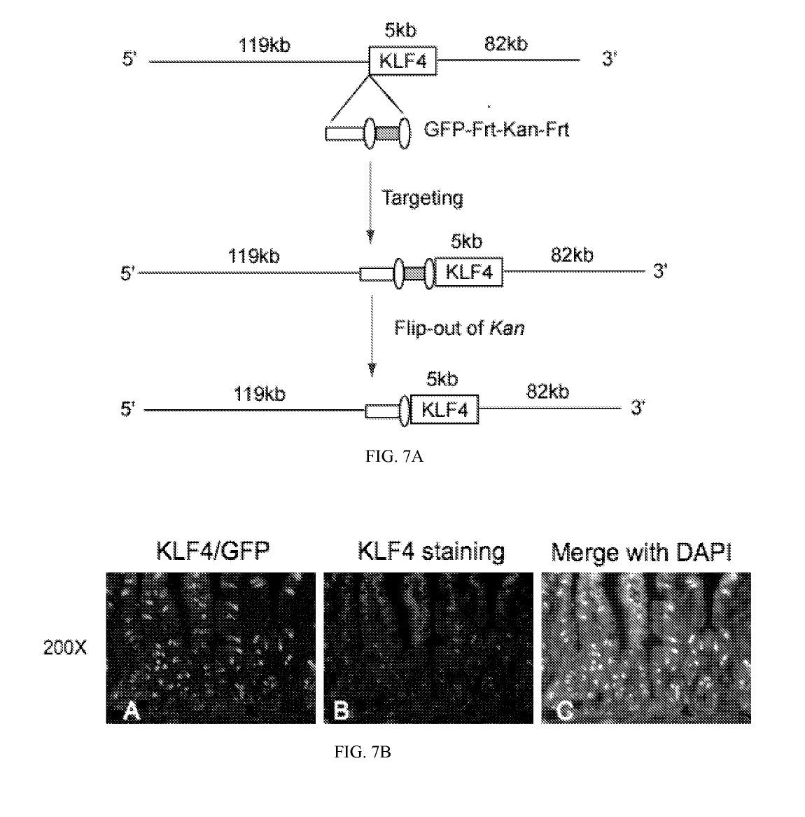

To study the in vivo regulation of KLF4, a KLF4/EGFP mouse model was generated using EGFP-containing construct as the transgene derived from a mouse KLF4 gene-containing bacterial artificial chromosome (BAC). This BAC clone (RP23-322L22) contains ˜119 kb 5′ and ˜82 kb 3′ fragments of KLF4 gene. In this transgene, EGFP was precisely inserted into the translational start site of KLF4 without deleting any endogenous KLF4 sequences, so that EGFP gene expression is under the control of the entire 5′ sequence of KLF4 gene. A diagram of construction strategy of the transgene was shown in FIG. 7A. Two potential mouse founder lines were generated after KLF4/EGFP was injected into fertilized mouse eggs of B6CBA/F1 background, and the lines showed similar levels of expression and both were backcrossed (6 times) to a wild type C57BL/6 background. In the KLF4/EGFP mouse, expression of EGFP was detected in the stomach, small intestine, colon, and testis in a pattern that matched closely gene expression of the endogenous mouse KLF4 (FIG. 7B). The specificity of the KLF4/GFP transgene was confirmed by colocalization of green signal of EGFP with that of KLF4 by immunostaining the small intestine and colon using an anti-KLF4 antibody.

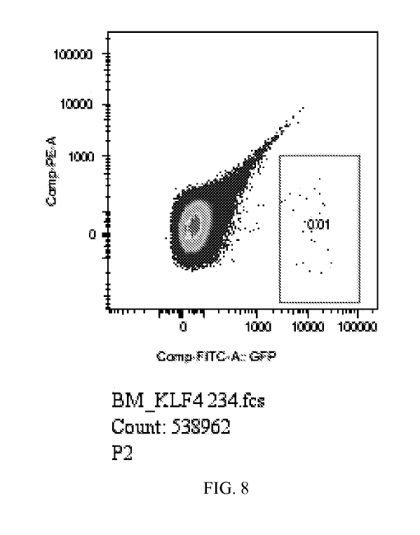

Given our previous studies showing KLF4 expression in a subset of BM mesenchymal cells, whole bone marrow was analyzed for GFP expression. In young mice (4-6 weeks old), EGFP signal was detected at a low level in the bone marrow by flow cytometry analysis (0.01-0.2%) (FIG. 8). These GFP cells were entirely negative for CD45 expression, and analysis of adherent MSCs also indicated that the KLF-GFP cells were mesenchymal in nature. Finally, the GFP (+) cells expressed endogenous KLF4, and showed similar levels of c-Myc, Sox2 and Nanog as our K19GFPMSCs but were negative for Oct3/4.

Identification of Bone Marrow Derived Mesenchymal Progenitor Cell Subset that can Contribute to Gastric Epithelium

A number of lines of evidence have pointed to possible plasticity by bone marrow derived cells. Studies in gender-mismatched bone marrow transplantation recipients in human patients have provided evidence for the contribution of bone marrow derived cells (BMDCs) to epithelial cells of the liver [1], lung [2, 3], and gastrointestinal tract [4-8]. Bone marrow transplantation experiments in mice have similarly shown the engraftment of BMDCs in a variety of epithelial tissues, where they presumably assist in the maintenance of the epithelium and help speed recovery from injury [2, 12-16]. Previously we showed clear evidence for the contribution of BMDCs directly to cancer. In mice reconstituted with labeled bone marrow, chronic Helicobacter felis (H. felis) infection induced repopulation of the gastric epithelium with BMDCs, followed by progression of these cells to intraepithelial cancer [17], indicating the potential role of BMDCs as cancer initiating cells. BMDCs have been also detected in human epithelial neoplasias in patients who have undergone a prior bone marrow transplantation, including renal cell carcinoma [9], colon adenoma [10], and lung cancer [10, 11].

In the majority of bone marrow transplantation studies that have been carried out in mice and humans, whole bone marrow cells or the enriched hematopoietic stem cell fraction were used to reconstitute the recipients' bone marrow. However, in most cases the infused donor cells were heterogeneous and it remains uncertain which cellular subset within the graft actually contributed to the epithelial lineage [8, 18]. In our earlier study, the mesenchymal stem cell (MSC) fraction of bone marrow was suggested as a possible source of BMDCs, since induction of gastric epithelial phenotype markers after treatment with gastric tissue extract was detected in the MSC fraction, but not in the HSC fraction [17]. Nevertheless, direct evidence of a possible MSC contribution to the gastric epithelium has yet to be established.

MSCs constitutes only a small fraction of the bone marrow [19, 20] and originally were characterized as colony-forming fibroblast-like cells [19, 21, 22] that in culture adhered to plastic and possess the ability to differentiate into osteocytes, chondrocytes, adipocytes, and myocytes [20, 23]. Thereafter, numerous studies in humans and animals reported unexpected differentiation in vitro into endodermal tissues such as neural cells [24], cardiomyocytes [25], pneumocytes [26], and hepatocytes [27]. More recently, a few studies have demonstrated pluripotency of MSCs in vivo [28-30].

In addition to their potential for differentiation into both mesodermal and endodermal lineages, some multipotent MSC subsets have been shown to express endoderm-associated genes, such as cytokeratins and Sox 17, along with known mesoderm-associated genes [29, 31, 32]. Cytokeratins are a family of intermediate filaments that are expressed in epithelial cells where they appear to be critical for the maintenance of the cytoskeleton [33]. Among the various cytokeratins, keratin 19 (K19) has been reported to be expressed in the progenitor zone of the epidermis [34] and the proliferative zone of the gastrointestinal tract [35-38], and is thought to be an early marker of epithelial cell lineage [39]. Transgenic studies in mice have demonstrated that K19 is expressed in the neck/isthmus region of the glandular unit, in the same area where gastric epithelial progenitors and committed precursor cells are believed to reside [35]. Importantly, bone marrow derived cells (BMDCs) were shown to consistently express K19 upon recruitment and engraftment in the stomachs of H. felis-infected mice [17].

The invention provides methods which assessed the ability of bone marrow-derived MSCs to differentiate toward a gastric epithelial cell lineage. The invention provides that K19 expression could be used to identify a subset of MSCs that possessed the ability to contribute to gastric epithelial cells. Results suggest that K19-expressing MSCs are absent from the normal bone marrow but induced by culture conditions in vitro, and in response to H. felis infection in vivo. Thus, K19-expressing MSCs possess plasticity along a gastric epithelial lineage and may potentially play a role in healing and repair of the gastric epithelium.

A number of studies have demonstrated that mesenchymal stem cells possess unexpected plasticity, and are able to differentiate across germ layer boundaries to give rise to epithelial tissues [28-30]. Previous reports by our group suggested MSCs as a possible candidate for the bone marrow-derived progenitor cells that give rise to cytokeratin (+) metaplastic cells of the gastric epithelium that develop in the setting of chronic H. felis infection [17]. In vitro studies indicated that MSCs, but not HSCs, could acquire an epithelial morphology and upregulate the expression of epithelial markers, such as cytokeratin 19, when exposed to gastric extract [17]. K19-expressing MSC sub-clones possess all of the gastric differentiating abilities of MSCs, and could give rise to gastric epithelial cells when injected into the adult mouse stomach or into early blastocysts. K19-expressing MSCs gave rise to K19 (−) MSCs, and were mobilized into the circulation by chronic H. felis infection.

Bone marrow-derived MSCs have been isolated and characterized in the past based on their fibroblast-like morphology, their ability to adhere to plastic, to proliferate in vitro and by their multi-lineage differentiation potential [40-45]. Nevertheless, no clear consensus has yet emerged on their expression of specific cell surface markers [40-42, 46]. In order to isolate MSCs in our studies, we cultured whole bone marrow on plastic tissue culture plates with medium containing 10% serum and passaged the adherent cells more than 5 times to eliminate contamination by hematopoietic and endothelial fractions, as confirmed by the CD45-, Flk1-, F4/80-phenotype of our MSC cultures. In addition, MSC phenotype was confirmed by their ability to form colonies in soft agar and by their ability to differentiate into osteocytes and adipocytes. Nevertheless, even cultured and enriched MSCs remain morphologically heterogeneous [46], and colonies derived from them are diverse in both appearance and differentiation potential [40, 46, 47].

Cytokeratin 19 (K19) is a marker of early epithelial progenitors [39], particularly in the stomach [35-38], and we noted upregulation of K19 when pooled MSCs were exposed to gastric tissue extract. We investigated K19 expression in individual MSC sub-clones and found that only a minority of individual clones spontaneously expressed K19 under basal culture conditions. Using a K19-GFP transfection approach, we selected for these K19-expressing clones and showed that they possessed greater gastric differentiation potential than K19 negative clones after treatment with gastric tissue extract in vitro. We obtained a greater percentage of mice with MSC-derived gastric epithelial cells after blastocyst injection of K19(+) MSC clones than the pooled parent MSC cultures, suggesting a greater potential by the K19 sub-clones to contribute into gastric epithelium in vivo. In addition, K19-GFP-positive cells gave rise to GFP negative cells with limited in vitro differentiation potential, indicating that the K19 positive cells are the more primitive fraction in the cell line.

While the K19-GFP MSCs appeared to possess some degree of longevity and self-renewal ability, the in vivo studies with these passaged cells raise some questions as to whether these cells are progenitors rather than true stem cells. In all of the stomach sections in which GFP positive cells were detected, positive cells were identified as isolated single cells scattered through out the gastric epithelium and not as clonal glands, nor as clusters. This is consistent with previous reports that bone marrow derived cells are detected as single differentiated epithelial cells in the uninjured gastrointestinal tract [8, 13, 30]. In addition, none of the scattered GFP positive cells showed expression of H/K-ATPase, indicating limited ability to differentiate into parietal cells, the most abundant type of differentiated cell in the gastric epithelium. A possible explanation for the lack of glands that are clonally derived from MSCs is that MSCs do not engraft into the stem cell niche under normal condition. However, it is also possible that our cultured MSCs have been converted from stem to progenitor cells during passage in culture, or else are simply limited in their multipotency. Our in vitro studies indicate that even after exposure to gastric extract and induction of gastric genes, they consistently express multiple gastric genes consistent with a progenitor rather than differentiating into a specific gastric lineage.

Multipotent mesenchymal stem cells have been reported to exist in the bone marrow [29, 30, 32, 49, 50], and these cells show expression of ES cell marker genes, such as Oct3/4 and Nanog [29, 30]. In this study, both the parental MSC cultures and the K19 (+) MSC clones expressed Nanog but not Oct3/4, and are negative for SSEA-1, suggesting that they are not identical with the previously reported multipotent mesenchymal progenitor cells. Finally, another possibility for the in vivo findings in the study is that the injected MSCs are simply undergoing cell fusion [51].

The expression of K19 was not detected in the mononuclear cell fraction which was freshly isolated from bone marrow. However, expression was detected in peripheral blood obtained from mice with chronic gastritis due to H. felis infection. The expression of K19 by both cultured bone marrow-derived MSCs and by MSCs in the circulation (but not the bone marrow) suggests that removal of MSCs from their normal bone marrow niche may in part trigger the upregulation of K19 and the epithelial progenitor program. In addition, MSC cultures were established more frequently from peripheral blood of H. felis-infected mice than from uninfected control mice, and bone marrow derived MSC cultures were established from the peripheral blood of bone marrow transplanted mice that were chronically infected with H. felis. The presence of MSCs in peripheral blood has been noted in several reports [52-54] and mobilization of bone marrow MSCs into peripheral blood under conditions of chronic inflammation or injury has also been described [55, 56]. Taken together, our results suggest that the expression of K19 was induced in bone marrow-derived MSCs after they are recruited into the circulation in response to chronic H. felis inflammation.

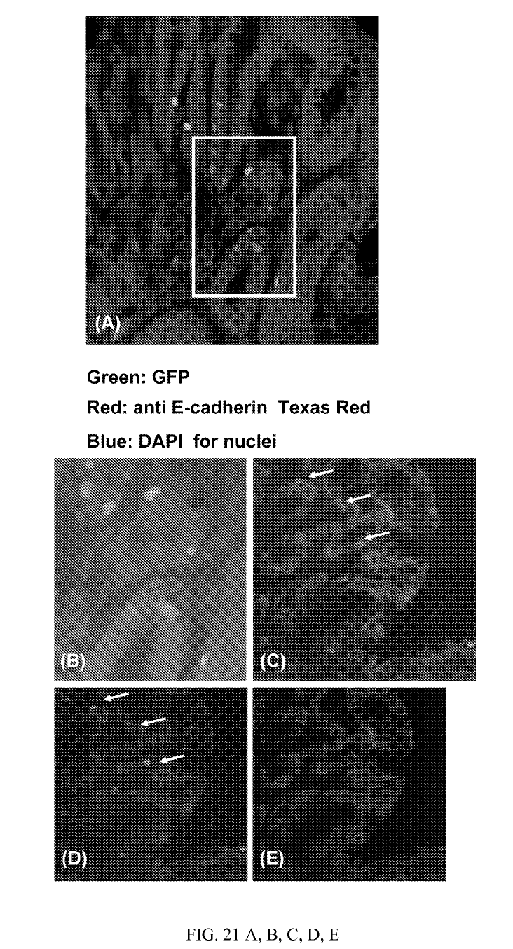

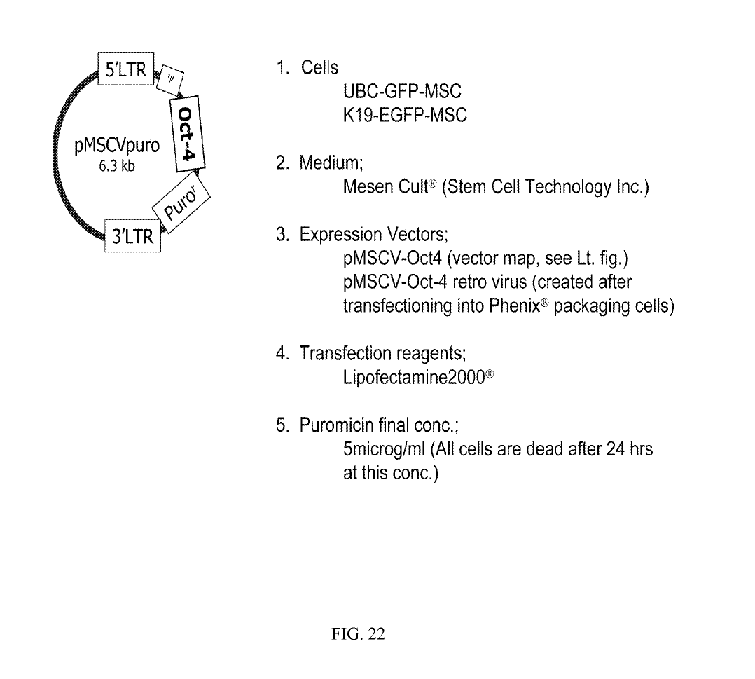

Results described herein, show that the chicken beta-actin eGFP MSCs (GFP-MSC) (hemizygous, C57BL/6-Tg (ACTB-EGFP) 10sb/J, JAX Stock number: 003291) showed primarily nuclear GFP signals when engrafted as epithelial cells, while the GFP expression in the epithelial cells derived from K19GFPMSC was quite robust. Chicken beta-actin eGFP signal were poorly expressed in gastrointestinal tract [57], and, in addition, eGFP can traffic back into nucleus following expression in the cytosol [58]. Nevertheless, confocal microscopy provided support for colocalization of GFP and E-cadherin and thus for epithelial expression of GFP (FIG. 21C-21E).

The invention provides that bone marrow derived MSCs can contribute to the gastric epithelium in vivo under experimental conditions, and that the K19 (+) MSC subset is most likely responsible for this contribution. We also detected K19 (+) MSC mobilization from into the peripheral blood in the setting of H. felis-dependent chronic gastritis. Although further studies will be necessary to determine whether there is any connection between MSCs and carcinogenesis, our data provide support for an MSC subset that could contribute to gastric epithelial regeneration and repair.

### EXAMPLES

### Example 1

### Conversion of MSCs into iPS Cells

Oct 4 overexpression effects on plasticity and mesenchymal differentiation patterns of K19-MSCs. In certain aspects, the invention provides MSCs expressing Oct3/4 and methods to investigate the multipotentiality of MSCs in which Oct4 is overexpressed. Currently, K19GFPMSCs and the corresponding parental MSCs show significant differentiation potential along multiple cell lineages but they are clearly not equivalent to ES cells or iPS cells in pluripotency or self-renewal capability. MSCs express good levels of KLF4 and c-Myc; they show lower levels of Sox2 and no detectable expression of Oct3/4. Although the levels of Sox2 in K19GFPMSCs are only 20-25% of that of ES cells, this level of c-Myc has proved to be sufficient for iPS formation [Nakagawa 2008]. Thus, the absence of Oct4 expression is the critical feature that limits the pluripotency of adult BM-derived mesenchymal stem cells. Oct4 is critical in the development of ES cells, since ES cells cannot be derived from Oct3/4 null blastocysts. Consequently, it is expected that (over)expression of Oct4 will alter the behavior and differentiation potential of MSCs. Should expression of Oct4 alone not prove sufficient, Sox2 will be (over)expressed in the same cell that expresses Oct4 .

Oct4 will be overexpressed in both K19GFPMSCs and parental unmodified MSCs. One strong advantage of using MSCs as the starting material is that they are relatively easy to stably transfect with plasmid vectors. For example, we have stably transfected murine MSCs with a constitutively active IKKbeta(EE)-construct and obtained several dozen stable transformants that show increased NF-kB activity. We have already obtained both plasmid and lentiviral expression constructs (iPSC Factor Expression Vector) for hOct 4. The lentiviral construct is based on the iPSC Factor Expression Vector and contains both an expression system for hOct4 and RFP under the control of the EF-1a promoter (SBI). The plasmid construct also contains a hygromycin selection marker, and can be transfected using the Lipofectamine 2000 transfection reagent, and stable lines generated by selection with hygromycin. Stable cell lines can also be generated after infection with lentiviral particles, and we will use viral supernatants from 293T cells infected with a mixture of viral plasmid and packaging constructs expressing the viral packaging functions and the VSV-G protein as previous described [Brambrink 2008]. In certain embodiments plasmid transfection is used to achieve forced expression of Oct4. In other embodiments, lentiviral infection is used to achieve forced expression of Oct4.

Twelve large plates of semi-confluent MSC and K19GFPMSC cells (approximately 100,000 each) in Mesencult Media (Stem Cell Technologies) will be transfected with Oct4 expression construct or infected with the lentiviral Oct4 construct. The cultures will be split 1:5 after 3 days of infection or transfection and the media changed to standard ES cell culture conditions of DMEM supplemented with 10% FBS and LIF as previously described. In the case of plasmid transfection, hygromycin (100-200 mg/ml) will be added to the cell culture media one day following the split. It is expected that over the next several weeks the morphology of the transfected MSCs will change, with the emergence of small, rounded cells forming in culture. After 21 days, we will pick single RFP-positive colonies (or yellow colonies in the case of K19GFPMSCs) and expand them on feeder MEFs in the absence of hygromycin. Some colonies will be picked and grown in Mesencult media which works well for our MSC cultures.

One aspect provides characterizing the stable Oct4-expressing MSC clones. We will first assess the morphology of the colonies, compared to the starting MSCs and also to mouse ES cells as previously described and to murine iPS cells that we have received from our collaborator Shinya Yamanaka (see letter). It has been reported that iPS cells can be identified to a large extent by morphology alone [Aoi 2008; Meissner 2007]. Colonies that appear morphologically indistinguishable from that of mouse ES cells and similar to earlier iPS cells will be designated iPS-MSCs, and hopefully these will be obtained for both K19GFPMSCs and parental MSCs. Prior to any systematic analysis, though, we will passage the cells for several additional weeks. Recent studies indicate that ectopic expression of Oct4 and other factors initiates a gradual reprogramming process in multiple cells that ultimately leads to pluripotency over a time period of several weeks [Brambrink 2008]. Oct4 overexpression has direct effects but also leads to upregulation of endogenous Oct4, and this endogenous upregulation correlates well with the iPS phenotype but requires several passages. Should we have a low yield of the ES-like phenotype from Oct4 overexpression initially, we can consider the use of the Oct4-EGFP reporter gene, which was created by methods know in the art, to follow this transition [Meissner 2007]. In addition, additional stable clones will be generated that overexpress both Oct4 and Sox2 using double transfection, or simultaneous lentiviral infection.

After passaging the morphologically most promising RFP(+) colonies, we will then begin analysis of the expression patterns and phenotypes of these cells. To select the cells with the greatest ES-like phenotype, we will carry out staining of selected colonies for alkaline phosphatase (AP), stage-specific embryonic antigen 1 (SSEA1) and Nanog at 3-4 weeks after transfection or transduction. Next we will examine in our colonies gene expression by RT-PCR (see FIG. 7) for Oct3/4, Sox2, c-Myc and KLF4, and levels will be compared to the parent cells, mouse ES cells and established murine iPS cells (courtesy of Shiny Yamanaka). Oct3/4 expression should be increased and detectable in all transfectants, and Sox2 should be increased in those cells transfected with the Sox2 constructs. While our MSCs and K19GFPMSCs show low-to-moderate levels of Nanog, we would expect to see an increase since Nanog is known to be a downstream target of Oct3/4. In the most promising colonies, expression of these factors will be confirmed by Western blot analysis, and we will also assess expression of other ES cell marker genes including Rex1, ECAT1, Cripto and Gdf3. We will assess the level of proliferation of thee colonies, since iPS cells tend to grow exponentially show the same proliferation rates as mouse ES cells [Aoi 2008]. Finally, iPS cells typically acquire an epigenetic state similar to ES cells, with demethylation of the Oct4 and Nanog promoters [Takahashi 2006; Maheraldi 2007]. Using methods known in the art, we will carry out bisulfite sequencing to assess the methylation status of the Oct4 and Nanog promoters, in comparison to the methylation status in our starting MSCs. Furthermore, global methylation studies (e.g. mSNP analysis) will be performed. In certain embodiments, valproic acid and/or 5′-azaC will be added for one week, which can increase the yield of iPS cells [Huangfu 2008]. It is expected that MSCs expressing Oct3/4, or Oct3/4 and Sox2, will show increased ES cell-like behavior and that starting K19GFPMSCs will yield a larger number of iPS-like clones. It is possible that K19 and GFP expression will be repressed in Oct4-expressing cells since K19 is not expressed that early in development.

We will then analyze the behavior of the Oct4 (and Oct4/Sox2) expressing cells in our MSC assays to determine if overexpression of Oct 4 or Oct4/Sox2 results in loss of the classical MSC phenotype. We will carry out colony forming units-fibroblast (CFU-F) assay, and differentiation assays, including adipocyte differentiation (assessed by Oil Red-O staining) and osteoblast differentiation (assessed with Alizarin Red staining) using Mesencult Stem Cell Media designed specifically for these assays. Finally, we will examine the ability of these cells to express gastric phenotype markers in vitro when co-cultured with gastric extract for 5 days (see FIG. 4). It is expected that the Oct4 expressing MSCs will lose their ability to differentiate into adipocytes, osteocytes and gastric cells under these defined conditions, reflecting their more basic embryonic stem cell phenotype. It is expected that Oct4-expressing MSCs will form embryoid bodies when grown in non-coated plastic dishes [Takahashi 2006]. ES cells are more pluripotent but in general harder to differentiate than many adult stem cells preparations. Overall, the expectations is the generation of MSCs that express Oct 4 along with c-Myc, KLF4 and Sox2, and that these cells will show more of an ES-cell phenotype in vitro.

Making of iPS Cells from K19-MSCs with Expression of One Gene (Oct4)

The invention provides methods to study Oct4-expressing MSCs in vivo and examine the possibility that forced expression of Oct4 in MSC clones as provided can lead to the generation of iPS cells. We will select the best candidates from the Oct4-expressing (or Oct4/Sox2-expressing) MSCs and/or K19GFPMSCs, based on morphology, Nanog expression and gene expression from the above stable cell lines.

To test pluripotency in vivo, we will transplant these cells (1×10⁶ cells) subcutaneously into the hind flanks of immunodeficient (nude or NOD/SCID mice) and observe the growth of these cells for up to 8 weeks. If tumors develop as expected, we will examine these lesions to determine if the tumors appear to be teratomas, containing various tissues from all three germ lines, such as neural tissue, muscle, cartilage, and gut-like epithelial tissues. This assay will thus determine if the putative iPS-MSCs are pluripotent and thus similar to ES cells and other iPS cells that have been reported. As a control for this study, we will use iPS-MEFs.

Next, we will inject several of the Oct4-expressing cell lines into blastocysts by microinjection, similar to our protocol for the K19GFPMSCs described above (see FIG. 5). We will test six (6) K19GFPMSC clones overexpressing Oct4 and six (6) beta-actin-EGFP MSC clones overexpressing Oct4 in this blastocyst injection model. Fifteen to twenty male cells will be injected into 129/Sv blastocysts (since our MSCs are derived from C57BL/6 mice). As a positive control for this study, we will again use established iPS-MEFs. We will determine if adult chimeric mice can be obtained from either of the Oct4-overexpressing groups of clones, our hypothesis being that the K19GFP-selected clones will perhaps show greater chimerism. In any case, we are confident that forced expression of Oct4 will increase the multipotency of our MSCs (which we have shown already display some multipotency) and possibly lead to pluripotency. Tail DNA will be analyzed from our chimeric mice for presence of the K19-EGFP, CBA-EGFP, or Oct4 construct. We will use SSLP analysis, along with immunofluoresence for GFP and RFP, to quantify the level of chimerism in various organs [Okita 2007]. A key area of analysis will be the testes in male mice, since we will then seek to establish whether germline transmission can be achieved with our Oct4-overexpressing MSCs. For the clones that show the highest level of chimerism in the testes, we will cross the male chimeric mice with Sv/129 females. While the F1 mice would be expected to show mostly agouti color, we would expect that half the F2 mice born from F1 intercrosses will show black coloring confirming germline transmission.

We will examine the tumorigenicity of mice derived from Oct4-expressing MSC clones. We will establish a cohort of 40-50 adult chimeras from 5 or more independent MSC clones, and we will follow these mice for up to 52 weeks. An increased rate of tumorigenesis has been reported in chimeric mice derived from iPS-MEF clones, while no increase in tumor formation has been reported in adult chimeras derived from iPS-Hep and iPS-Stm clones. We will also assess the incidence of perinatal death of chimeric mice, which has been noted to be increased in some iPS clones [Aoi 2008].

iPS cells derived from bone marrow mesenchymal cells that endogenously express KLF4. We will explore further the expression of KLF4 in a subset of bone marrow mesenchymal cells to determine the overlap of these cells with K19GFPMSCs, and also investigate whether bone marrow cells selected on the basis of KLF4 expression can also be converted efficiently into iPS cells with expression of 1-2 genes, Oct4, or Oct4 and Sox2. The availability of our KLF4-EGFP transgenic mice, which showed GFP expression in a subset of bone marrow mesenchymal cells, has afforded us the ideal opportunity to investigate this “stem cell marker” in bone marrow cells. We have demonstrated that they have a gene expression profile (KLF4+c-Myc+Sox2+Oct4−) remarkably similar to that of K19GFPMSCs, and thus can be isolated without prior culture. It is expected that the KLF4-GFP subset of bone marrow cells does represent the relevant subset of cells that are best suited as starting material for iPS generation. Another goal will be to find additional markers for selection independent of the transgene, we will begin by testing their plasticity and the effect of forced expression of Oct4.

KLF4-GFP+ cells will be isolated using two approaches. GFP+ fraction from the bone marrow of KLF4-BAC-EGFP transgenic mice (C57BL/6 background) will be sorted by FACS, and culture these cells directly in Mesencult media (SCT) on plastic dishes. In another embodiment, MSCs (the adherent fraction) will be isolated from the bone marrow of KLF4-EGFP mice in standard fashion, flushing the femurs of mice and plating BM cells (106 cells/cm2) and removing nonadherent cells after 24 hrs. The adherent cells will be cultured and the GFP+ colonies picked for further passaging. After several passages, we will test their plasticity as MSCs as described above. The cells will be tested in CFU-F assays, and in adipocyte and osteocyte differentiation assays using define media. The cells will be examined for K19 expression and for gastric differentiation using co-culture with gastric extract and expression of defined markers (H/K-ATPase, TFF2, MUC5AC, and chromogranin A). We will passage several times the KLF-EGFP cells and assess GFP expression after each passage to assess the percentage (%) of cells that remain GFP positive. The GFP sort will be repeated after several passages, and confirm that the GFP(+), KLF4(+) cells give rise to GFP(−) cells. The expectation is that the KLF4(+) subset represents a more primitive progenitor population, but may or may not express K19 and show easy differentiation along a gastric lineage similar to the K19GFPMSC subsets. However, it will be important to define the relationship of this BM mesenchymal subset relative to our pooled MSCs and the K19GFPMSC subsets.

Next, we will carry out forced expression of Oct4 in these KLF4-GFP cells. We will use plasmid or lentiviral vectors that also express RFP as described above, and select out clones using a similar set of approaches. Stable cell lines will be generated using hygromycin with plasmid constructs or through lentiviral vectors. After 21 days of selection, cells will be expanded on feeder MEFs (in the absence of hygromycin) and grown in Mesencult growth medium. Selection of colonies will be based initially on RFP expression, and later on morphology, specifically clones that show a morphology most similar to ES cells. After passing morphologically most promising RFP(+) colonies, we will then begin our analysis of the expression patterns and phenotypes. On selected colonies, we will carry out staining for alkaline phosphatase, SSEA-1 and Nanog at 3-4 weeks after transfection or transduction. This will be followed by a complete RT-PCR assessment of Oct4, Sox2, c-Myc, KLF4 and Nanog, along with other stem cell genes listed above. In certain embodiments, Sox2 will be overexpressed along with Oct4 into KLF4GFPMSCs. Oct-4 expressing clones, or clones expressing Oct4 and Sox2, will be compared to the parent KLF4GFPMSC clones in models of subcutaneous injection (into NOD/SCID mice) and after blastocyst injections. It is expected that Oct4 overexpression in these cells will result in increased ES cell-like behavior and possibly in the generation of iPS cells.

Materials and Methods

Cell Lines: K19GFPMSC Lines #3, #4, and #5 are grown in murine mesenchymal medium with murine mesenchymal supplements (Stem Cell Technologies, Vancouver, Canada). The cells became confluent within 2-3 weeks at 37 C in the humid air containing 5% CO2. Adherent cells were detached by 0.25% Trypsin and 0.02% ethylene diaminetetraacetic acid (EDTA) at 37 C for 2 min and subsequently passaged in the ratio of 1:3 to achieve the required number.

Human MSCs are grown in human mesenchymal medium with human mesenchymal supplements, and under conditions suitable for culturing human MSCs. Sequences of various genes and proteins used in the methods of the invention are available from NIH or GenBank.

Vectors: Lentiviral expression constructs for Oct3/4 and Sox2 (piPSC constructs) were obtained from System Biosciences (SBI) and include RFP expression under the EF1alpha promoter. Lentivirus is prepared in standard fashion using 293T cells in IMDM media and a combination of envelope plasmid (pVSVG), packaging plasmid (pMDLg/Prre), splcing regulator (pRSV-Rev) and our lentivirus DNA (piPSC).

Oct3/4 and Sox2 plasmid constructs: hOct4 (also referred to as Oct3/4)and hSox2 sequences are available from NIH or GenBank. Plasmids carrying Oct4 or Sox2 can be constructed by methods known in the art. Certain plasmids which can be used in the methods of the invention are available from addgene.org

Transfection of Oct3/4 and luciferase assays: Mouse mesenchymal stem cells (mMSC) are cultured in Mesencult MSC basal medium (Stemcell Technology, USA) as manufacture's instructions, and cells were seeded into 6-well plates and transfected 24 hours later with Oct3/4 expression plasmids containing puromycin selection markers using Lipofectamine 2000 transfection reagent (invitrogen, Carlsbad, Calif.). Stable pools of Oct4-expressing MSCs are selected by growing cells in 200 mg/ml hygromycin for 6 weeks.

Induction of expression of gastric phenotype markers in vitro. Stomachs are removed from wild type C57BL/6 mice at age of 8-12 weeks and gastric tissue paste is made by mortar and pestle. The paste from one stomach is mixed with 10 ml of Mesencult Stem Cell Medium and incubated at 4 degree for 24 hours, centrifuged and the supernatant filtered using 0.2 micro m membrane. The cells were cultured with the medium containing gastric extract for 5 days at 37 oC in the humid air containing 5% CO2.

Blastocyst injection of MSCs. Blastocyst injections are performed in the Transgenic Core Facility of Columbia University Medical School. Day 3.5 blastocysts are extracted from the uteri of C57Bl/6J pregnant females. Day-3.5 blastocysts were injected with 10 to 15 GFP labeled MSCs and implanted in uteri of Swiss Webster pseudopregnant females.

Quantitative real-time PCR analysis. Total RNA is isolated using Trizol (Invitrogen, Carlsbad, Calif.). First-strand cDNA is synthesized using the SuperScript First-Strand Synthesis System with SuperScript III reverse transcriptase (Invitrogen). The cDNA generated is used as a template in real-time PCR reactions with the QuantiTect™ SYBR® green PCR kit (QIAGEN, Maryland, Mass.) and products run on an ABI Prism 7300 Sequence Detection System (Applied Biosystems, Branchburg, N.J.). Each PCR run includes a 15-min activation time at 95° C. The three-step cycle includes denaturing (94° C., 15 s), annealing at 55° C. and extension at 72° C. mRNA quantities are analyzed in duplicate, normalized against GAPDH as an internal control gene. Results are expressed as relative gene expression using the delta delta Ct method.

Flow cytometry. Adherent cells are detached by 0.25% Trypsin and 0.02% EDTA at 37 C for 2 min, washed with blocking buffer (PBS w/1% fetal bovine serum, FBS), and suspended in the same buffer. Then cells are incubated with labeled antibodies at 1 micro g/1,000,000 cells for 30 minutes at 4° C. PE-conjugated rat IgG2a antibody (Jackson ImmunoResearch, West Grove, Pa.) was served as isotype controls. The cells are analyzed by using BD LSRII (Becton, Dickinson). 4′,6-diamidino-2-phenylindole (DAPI) was added to exclude dead cells.

### Example 2

### Mesenchymal Stem Cells and Gastric Epithelium

Establishment of Bone Marrow-Derived MSC Cultures and Induction of Gastric Phenotype Markers Following Treatment with Gastric Tissue Extract.

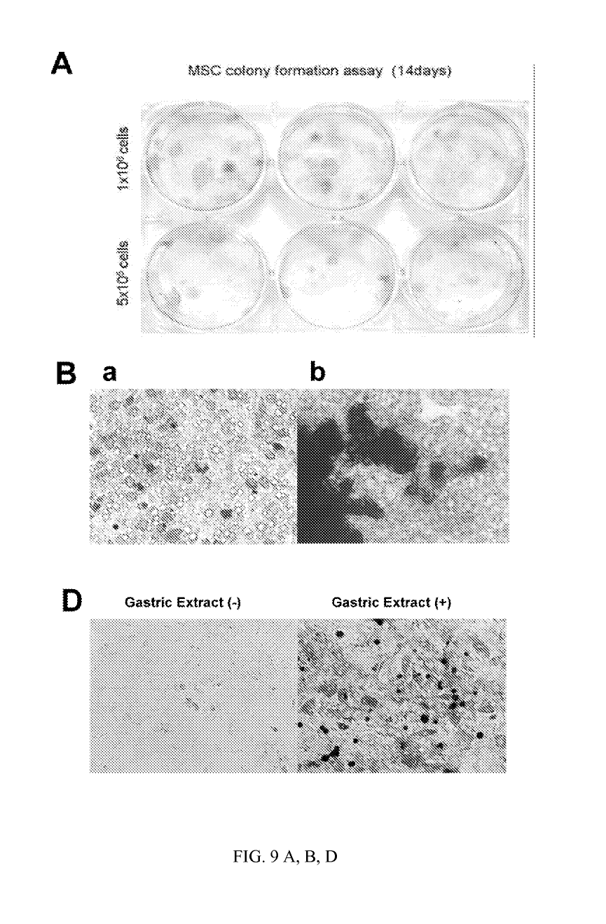

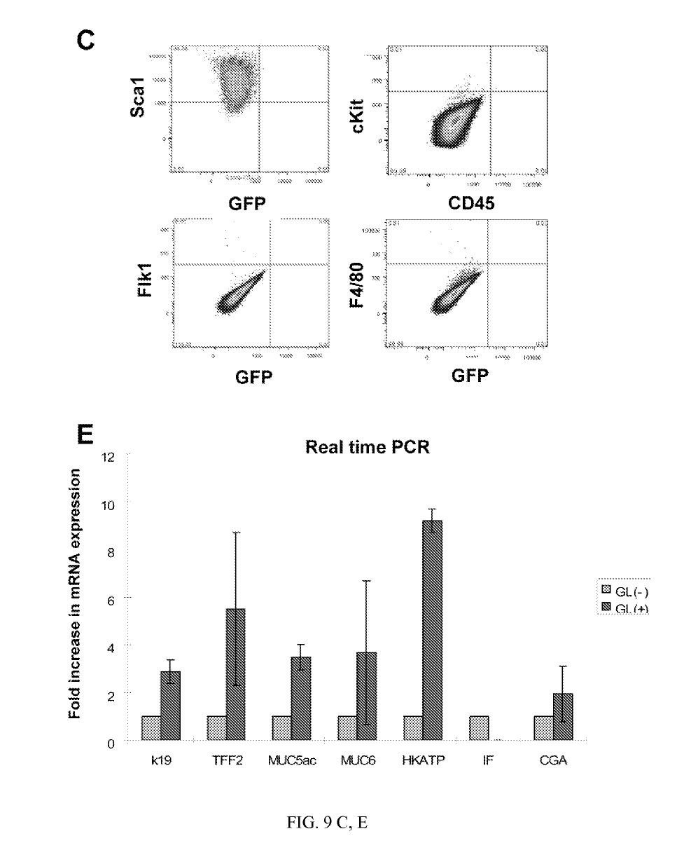

We established MSC cultures from whole bone marrow from mice as previously described based on their ability to adhere to plastic tissue culture dishes [19-23]. Non-adherent cells were removed and the primary cultured MSCs became confluent within 2-3 weeks and grew exponentially for more than 15 passages without signs of senescence or differentiation. After 5 passages, the pooled MSCs demonstrated the abilities of colony formation (FIG. 9A) and the ability to differentiate into both adipocyte and osteocyte lineages under previously defined conditions (FIG. 9B). Flow cytometry (FACS) analysis of these primary MSC cultures revealed that majority of the cells expressed Sca-1 (94.4%), but not CD45, c-kit, or Flk-1.

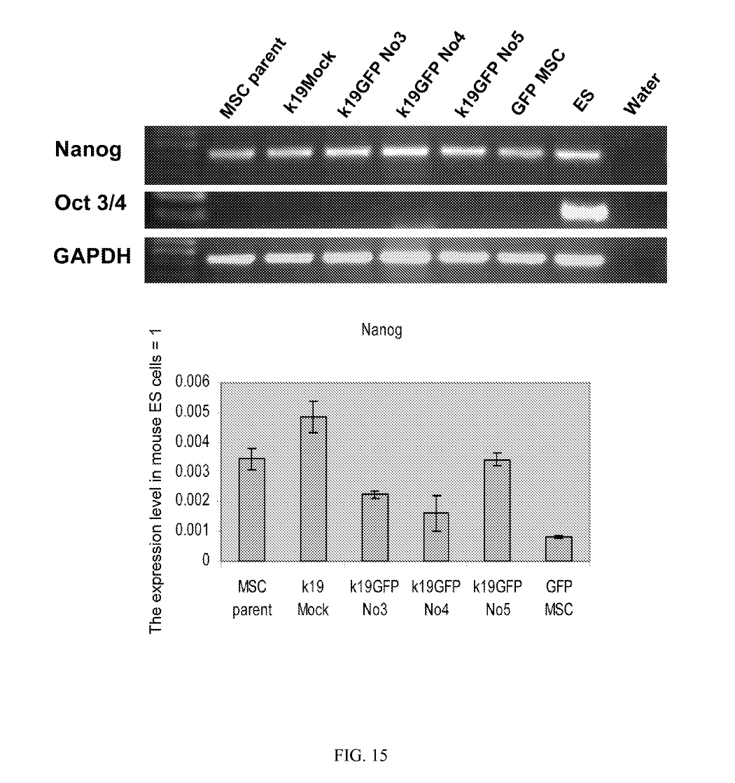

Since previous reports have suggested that a subpopulation of cultured MSCs exhibit multipotency in association with expression of embryonic stem cell markers [29, 30], we examined the expression of ES cell markers such as Nanog and Oct-3/4. Low levels of Nanog, but not Oct 3/4, expression were detected in our cultured MSCs (FIG. 15). Following treatment with gastric tissue extracts [see Materials and Methods], the cultured MSCs altered their morphology from spindle-like fibroblastic to oblate or irregular appearance under phase contrast microscopy (FIG. 9D). In addition, treatment with gastric extract resulted in increased expression of gastric epithelial phenotype markers such as K19, TFF2, MUC5AC, MUC6, H/K-ATPase, and chromogranin A in MSCs (FIG. 9E).

Identification and Isolation of Specific MSC Clones that Express Cytokeratin 19 (K19).

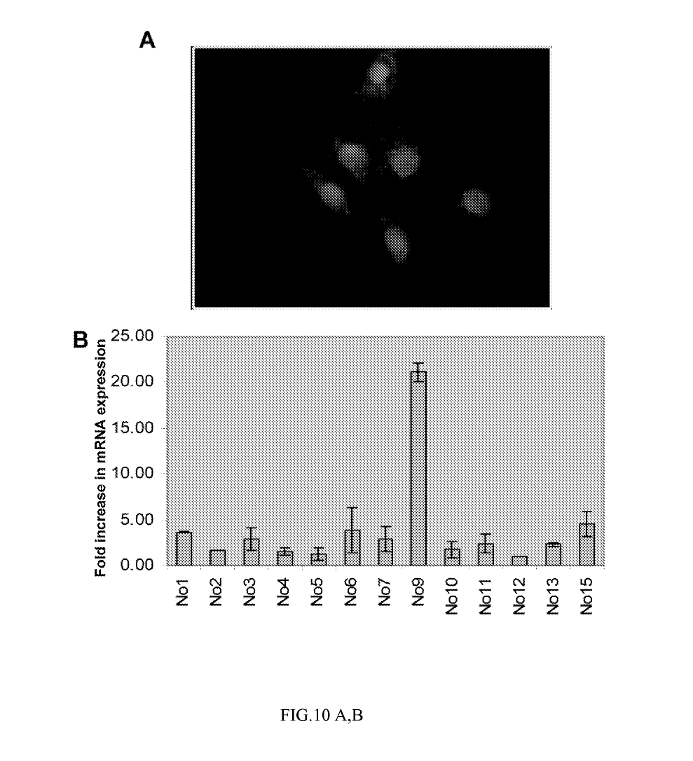

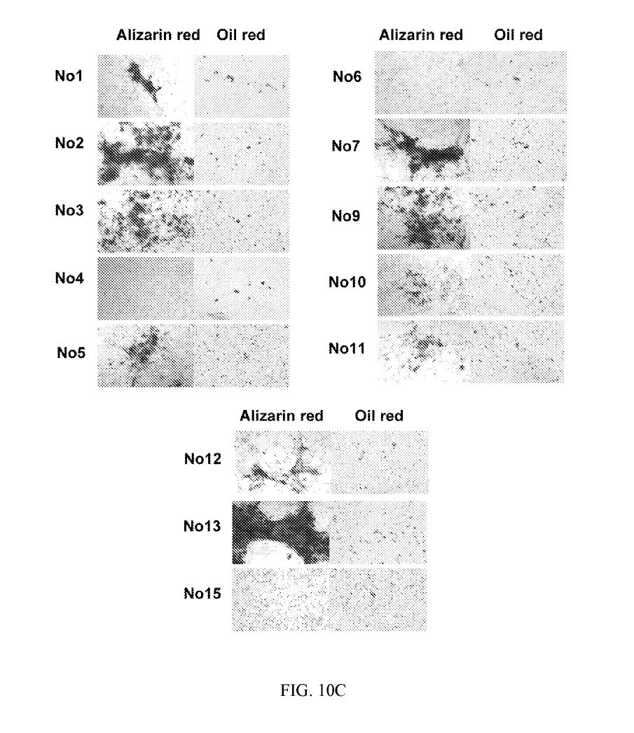

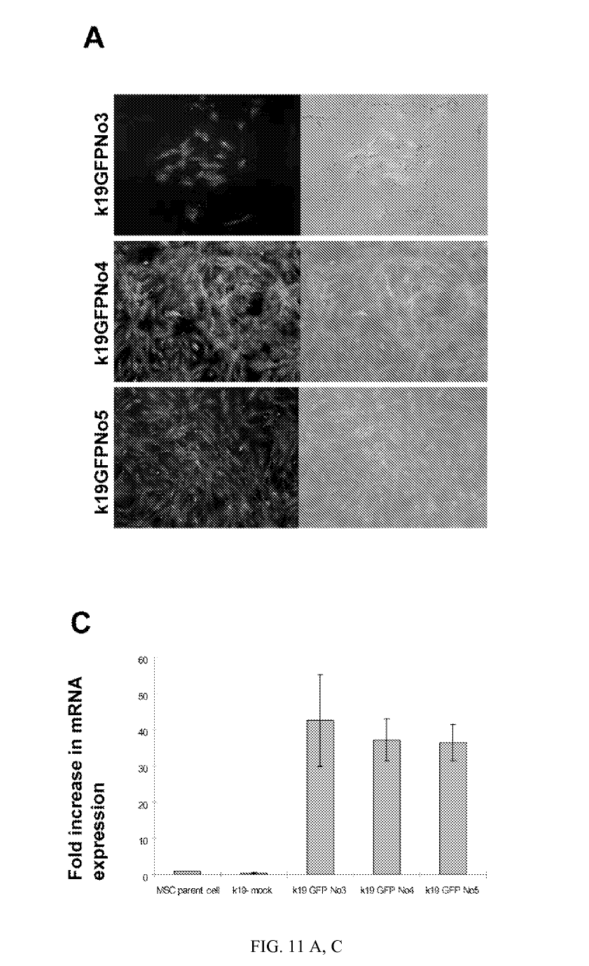

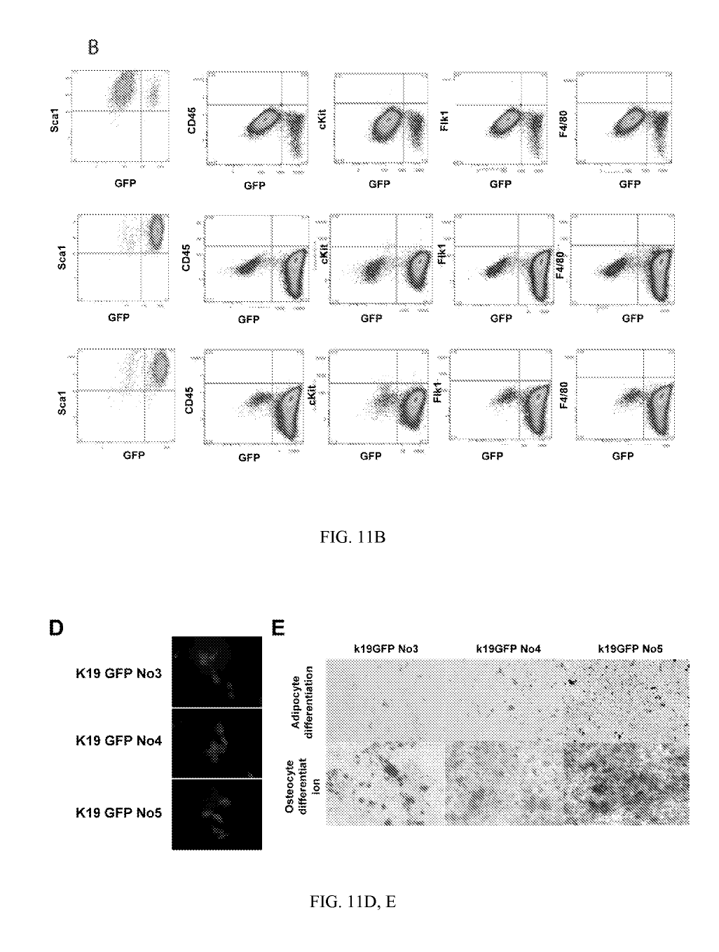

We found that K19 was expressed at a low level or in a small subset of cultured MSCs (FIG. 9E). Immunofluorescence staining confirmed that a small number of cultured MSCs expressed K19 (FIG. 10A). Individual MSC colonies were isolated from primary cultures, expanded and then tested for K19 expression by RT-PCR. High levels of K19 mRNA expression could be detected in approximately one out of 13 subclones from 1 mouse (FIG. 10B). This implied that, although K19 expression was present or could be included in a small subset of MSCs, K19+ cells could expand clonally and then be enriched for K19 expression. Most of these subclones (which were not truly clonal but colony picks from the original MSC cultures), including the clone with highest level of K19 expression, retained the ability to differentiate into osteoblast and adipocyte lineages under appropriate culture condition (FIG. 10C).

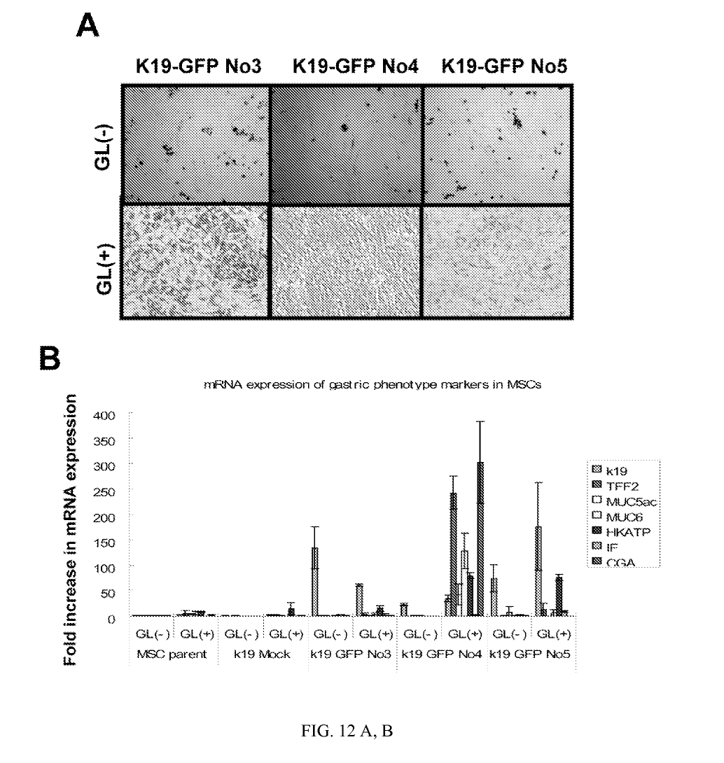

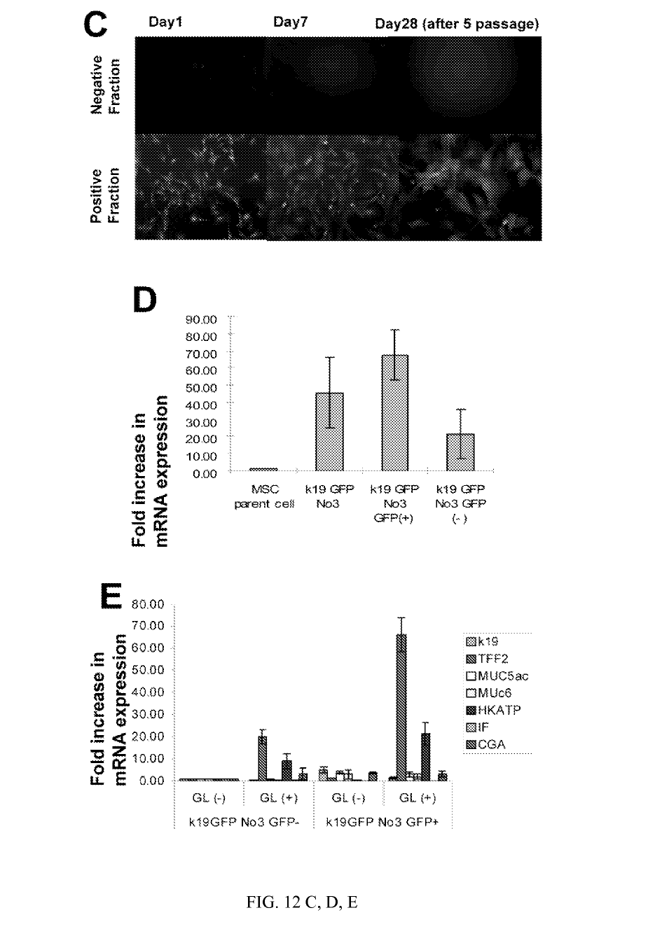

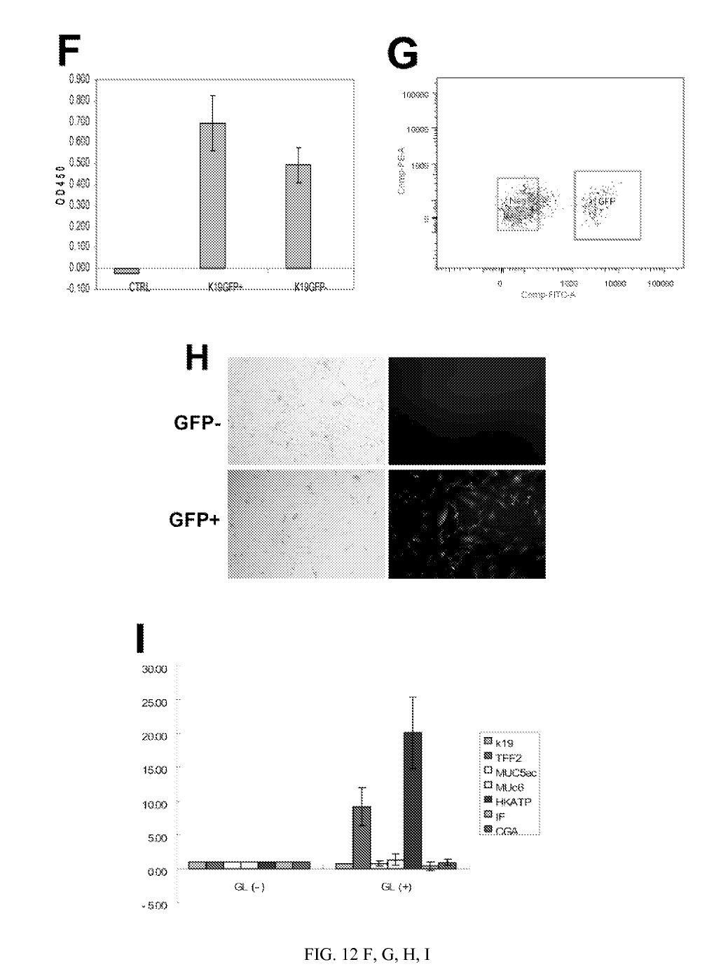

In order to isolate the minority of MSC clones that express K19, primary cultured MSCs were transfected with a K19-EGFP expression vector and stable clones were selected following G418 treatment. Fluorescence microscopy revealed 3 of 11 isolated clones from the same parent MSCs to be positive for GFP expression (FIG. 11A), and these clones (K19GFPMSC) were designated K19GFP No 3, No 4, and No 5. Flow cytometry revealed that the percentage of GFP positive cells was 10.0%, 96.2%, and 78.6% for K19GFP No 3, No 4, and No 5, respectively (FIG. 3B). Real time PCR analysis showed 40- and 35-fold increases in K19 mRNA expression level in K19GFP No 4 and No 5, respectively, compared to the parent MSCs (FIG. 11C). The average K19 mRNA expression of K19GFP No 3 was approximately 42-fold (FIG. 11C) compared to the parental MSCs, and the average K19 expression in clone No 3 did not show a statistical difference from the other 2 clones despite the lower percentage of GFP (+) cells. K19 mRNA expression in K19GFP No 3 was primarily due to the high level of K19 expression in the GFP+ fraction, with the GFP(+) cells showing 66-fold elevated expression compared to 16-fold for the GFP(−) cells (FIG. 12D). Expression of K19 protein in the three stable MSC clones was confirmed by immunofluorescent staining (FIG. 11D). FACS analysis revealed that expression of a variety of cell surface markers (including Sca-1, CD-45, c-kit, Flk-1, and F4/80) in the K19GFPMSC clones was roughly similar to that in the parent MSCs (FIG. 11B, see also FIG. 9C). All three K19GFPMSC clones expressed Nanog, but not Oct3/4 (FIG. 15), and retained the ability to differentiate into adipocyte and osteocyte lineages in culture (FIG. 11E).

Treatment with Gastric Tissue Extract Up-Regulates Expression of Gastric Phenotypic Markers in K19 Positive MSCs.

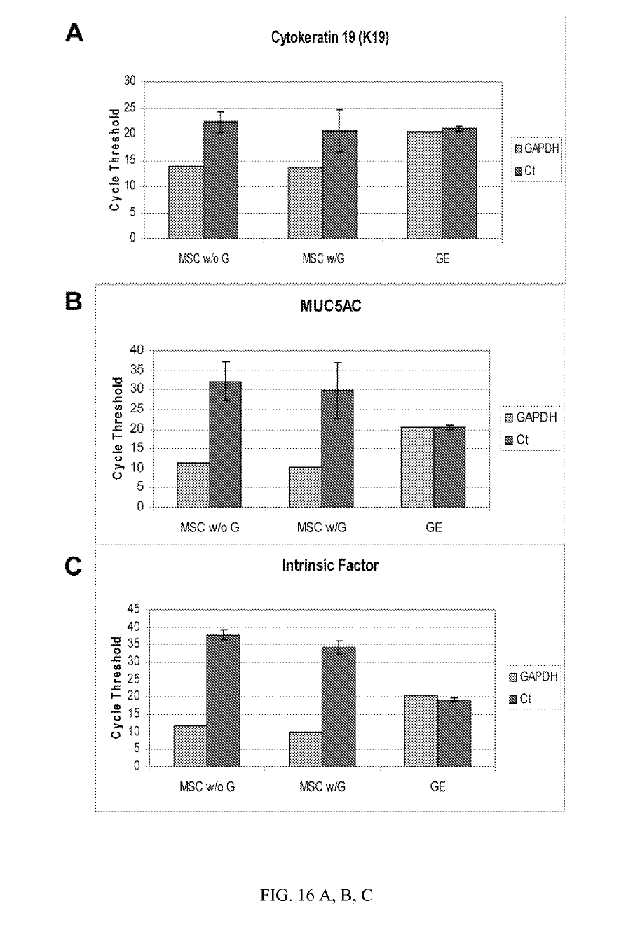

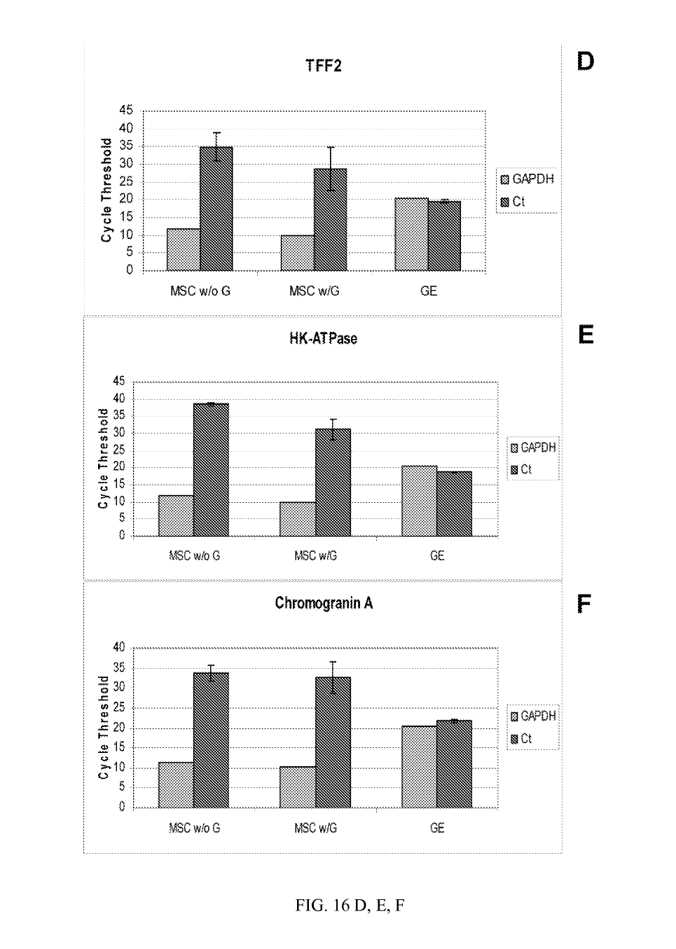

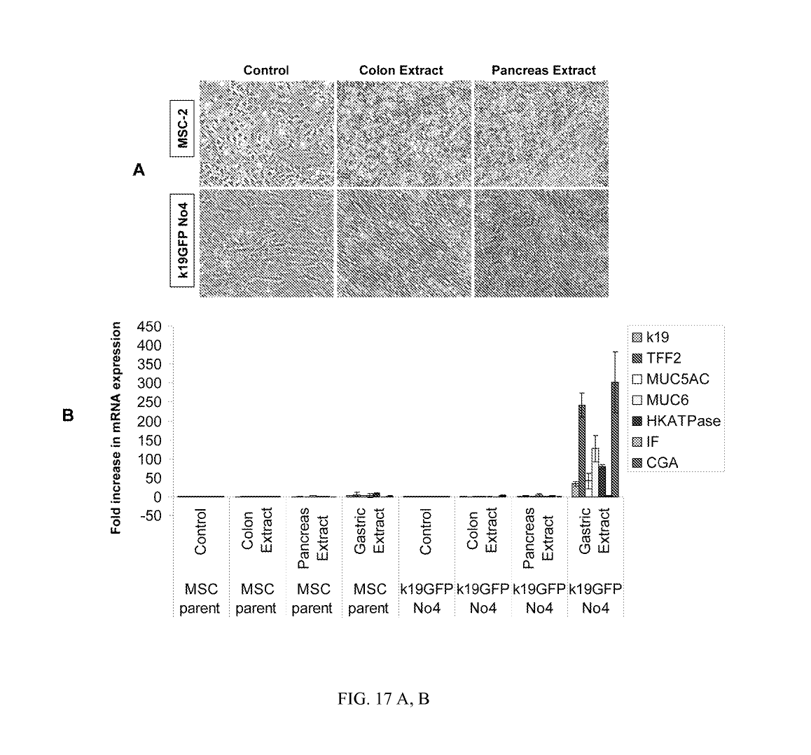

Following five days of treatment with gastric tissue extract, K19GFP MSCs altered their morphology from spindle-like to oblate or irregular shape (FIG. 12A), similar to what had been observed in the parental MSCs. In addition, after 3 separate experiments, the K19GFPMSC clones showed significant increases in mRNA expression of gastric phenotypic markers, with up to 60-, 300-, and 170-fold increases in K19GFPMSC clones No 3, No 4, and No 5, respectively. In contrast, less than 10-fold increases were observed in the parental MSCs and mock transfectants (FIG. 12B). This suggests that the potential of MSCs to express the gastric phenotype may be related to their level of K19 expression. Although the induced expression of gastric phenotypic mRNAs in K19GFP MSCs treated with gastric tissue extract is much lower than in gastric tissue, the increases seen with exposure to gastric extract are quite reproducible (FIG. 16). In contrast, treatment of K19GFPMSCs clones with either colonic or pancreatic tissue extract did not induce up-regulation of gastric phenotype markers in the K19GFPMSC clone No 4 (FIG. 17).

Progenitor-Like Characteristics of the K19 GFP (+) MSCs

After twenty-eight (28) days of culture, sorted GFP (+) cells gave rise to both GFP(+) and GFP (−) cells, while GFP (−) never gave rise to GFP (+) cells under the same conditions (FIG. 12C). BrdU assays showed the GFP (+) cells exhibit higher proliferation rates compared to GFP (−) MSCs (8 samples each, unpaired Student's t-testp=0.0029) (FIG. 12F). Real-time PCR showed a higher basal level of K19 mRNA expression in GFP (+) MSCs compared to GFP (−) MSCs (FIG. 12D). Following treatment with gastric tissue extract, the K19-expressing GFP (+) fraction showed greater up-regulation of gastric phenotypic markers, such as TFF2 and H/K-ATPase, compared to the GFP (−) fraction (FIG. 12E). To further address the nature of single GFP (+) or GFP (−) cells, we performed single cell sorting (FIG. 12G) and these studies showed that single sorted cells could divide and generate colonies (FIG. 12H). After treatment with gastric tissue extract, the overall pattern of gastric phenotype markers expression and up-regulation was similar between colonies from the single sorted and pooled GFP (+) and GFP(−) MSCs, although the difference between GFP(+) and GFP(−) MSCs remained significant in this analysis colonies from single sorted clones (FIG. 12I). In addition, isolation of single colonies from GFP(+) MSCs treated with gastric extract demonstrated the same pattern of genes expression when re-incubated with gastric extract. Taken together, these data show that GFP(+) MSCs have greater potential to up-regulate a couple of gastric phenotype markers, without specifying a particular differentiated gastric epithelial lineage. The results suggest that the K19-expressing MSCs may represent a progenitor cell fraction.

In vivo Differentiation of K19-Positive MSCs into Gastric Epithelial Cells.

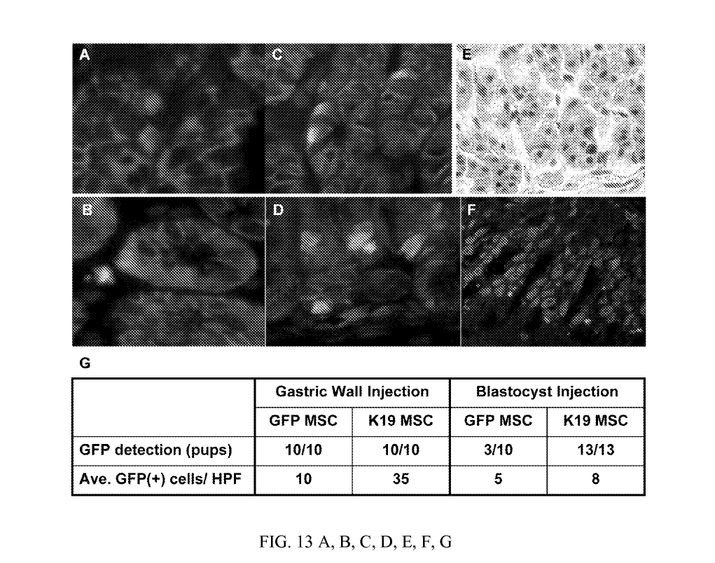

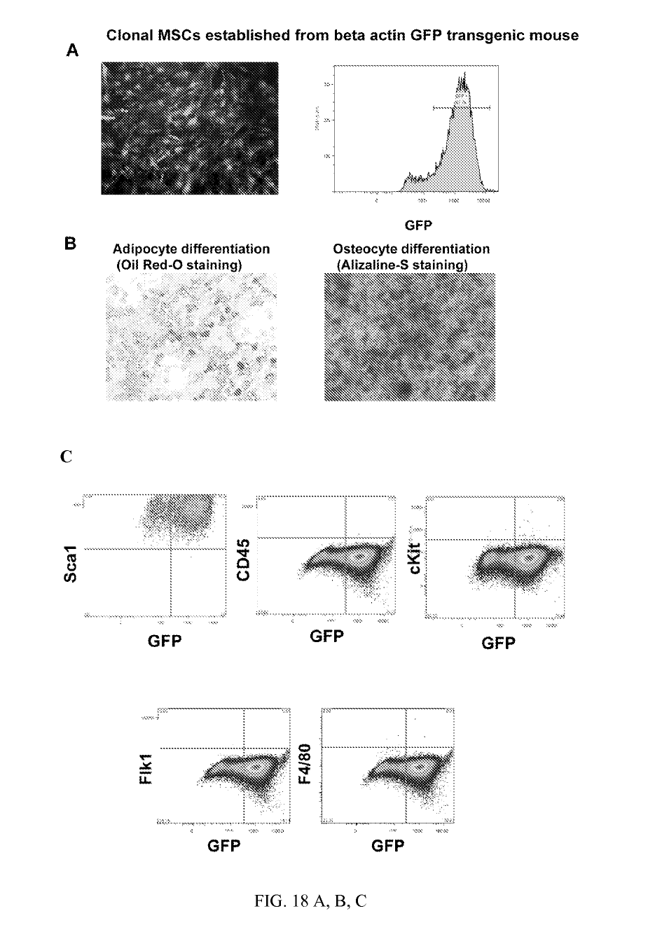

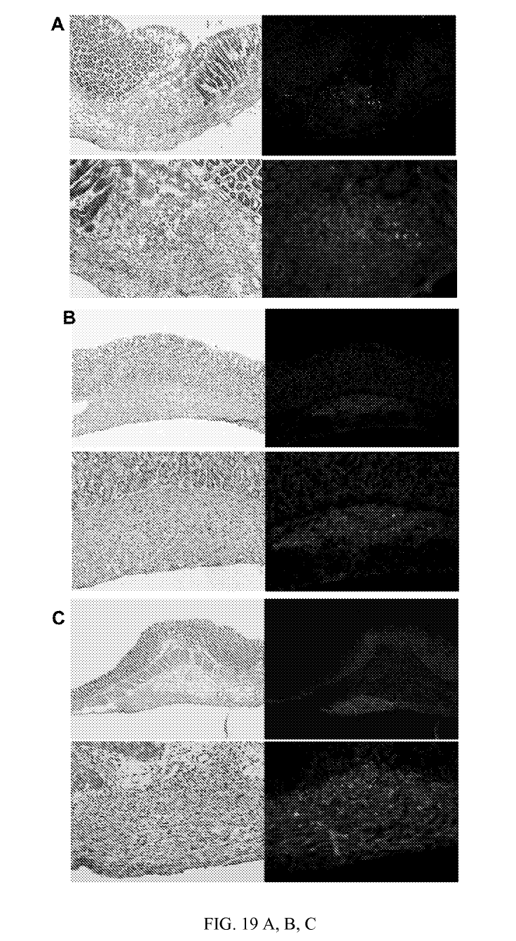

To test their differentiation ability in adult animals, two hundred thousand (200,000) cells from either No 4K19GFPMSC or control GFPMSC, the latter was established from the bone marrow of chicken beta actin EGFP transgenic mice (FIG. 18, see also FIG. 9C), were injected directly into stomach wall of C57BL/6 wild type mice. Twenty-four hours after injection of control GFPMSC cells, GFP positive cells were found distributed in the mucosa, submucosa, and subserosa of stomach (FIG. 19). A similar pattern was observed for the K19GFPMSC cells. Two weeks after injection of No 4 K19GFPMSCs, GFP positive cells could be detected scattered through the gastric epithelium, with many showing expression of the epithelial specific marker, E-cadherin, on their cell membrane (FIG. 5A). Stomach sections from mice injected two weeks earlier with the control GFPMSCs showed relatively fewer cells but also a number that expressed were positive for both GFP and E-cadherin markers in the gastric epithelium (FIG. 13B).

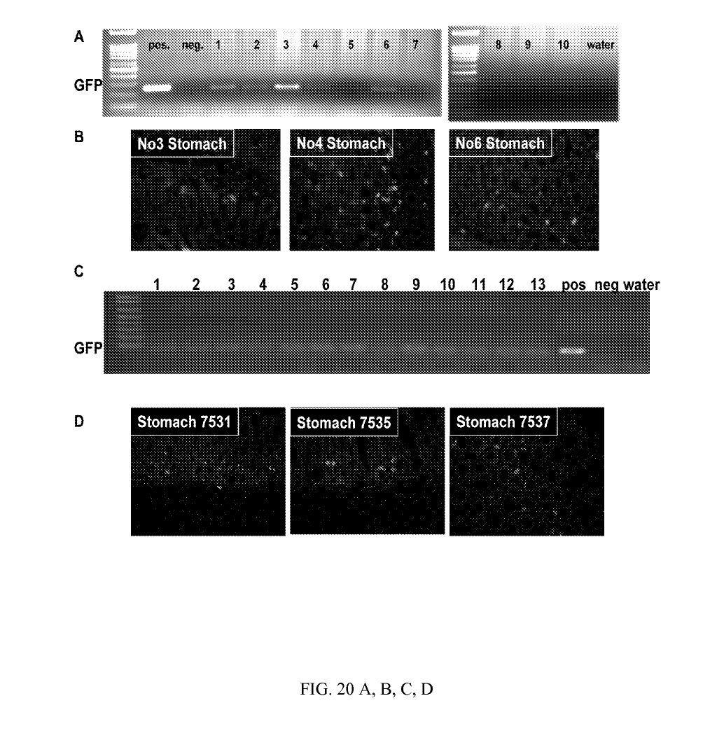

To test the differentiation ability of these cells under embryonic conditions, we performed blastocyst injections of GFP-labeled MSCs. Following blastocyst injection of control GFPMSCs, GFP DNA sequence were detected by PCR in tail DNA from 6 of 10 chimeric offspring (FIG. 20A), and analysis of gastric sections from 8 week old mice showed GFP positive cells in the gastric epithelium in 3 of 10 animals (FIG. 20B). Some of the GFP+ gastric cells also showed E-cadherin positivity (FIG. 13C). In comparison, GFP (+) cells could be detected in the gastric epithelium of all 13 pups derived from blastocyst injection of No 4 K19GFPMSCs, (FIGS. 20C and 20D) with many GFP (+) E-cadherin (+) cells were distributed throughout the gastric epithelium (FIG. 13D). We confirmed the localization of GFP inside and E-cadherin on the membrane of the same cells under confocal microscopy (FIG. 21). Immunofluorescent study against GFP protein with anti GFP antibody further confirmed engraftment of injected MSCs into gastric mucosa (FIG. 13E). To track the differentiation of these engrafted MSCs, we performed immunofluorescent staining for H/K-ATPase, intrinsic factor, chromogranin A, MUC5AC and MUC6, but we didn't find any GFP positive cells co-expressing either of these markers in any of the sections (FIG. 13F). The quantification of GFP detection rate, based on the number of animals positive for GFP expression as well as the number of GFP(+) cells/high power field (HPF) (average of 10 HPFs), from both the gastric wall injection study and the blastocyst injection study, are summarized in FIG. 13G. Overall, it appears that K19GFP MSCs show an engraftment rate that was equal to or greater than that of pooled GFP MSC.

K19 Positive MSCs in Peripheral Blood of Mice with Chronic H. felis Infection.

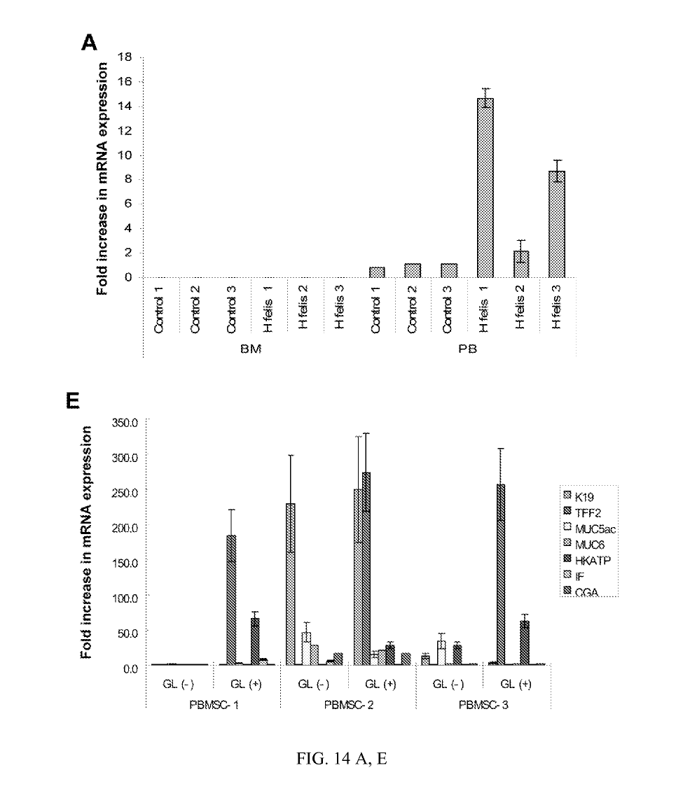

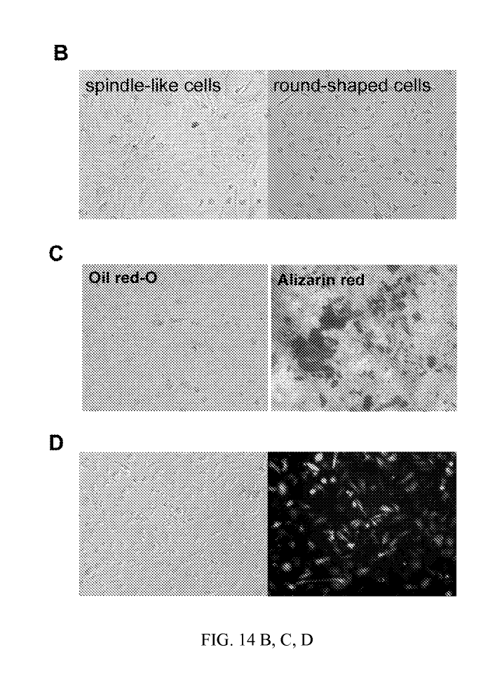

Although real time RT-PCR showed no expression of K19 mRNA in fresh bone marrow cells of mice, regardless of chronic H. felis infection, K19 mRNA expression was detected in mononuclear cell fraction of peripheral blood of mice and was highly up-regulated by chronic H. felis infection (FIG. 14A). Furthermore, when the mononuclear cell fraction of peripheral blood from mice with H. felis infection was seeded into plastic plate with MSC culture medium, spindle-like fibroblastic cells proliferated exponentially, while many of round-shaped cells did not grow and lost from the culture after passage (FIG. 14B). We designated the spindle-like fibroblastic cells as peripheral blood derived MSCs (PBMSCs) since they showed adipocyte and osteoblast differentiation under appropriate culture condition (FIG. 14C). PBMSCs were established from 5 of 13 mice (38.5%) that were chronically H. felis-infected, while they were established from only 1 of 14 (7.1%) age matched uninfected control mice. Most of PBMSCs were GFP-positive when they were established from mice which received bone marrow transplantation from chicken beta actin EGFP donor mice (FIG. 14D), suggesting the bone marrow origin of PBMSCs. In addition, PBMSCs showed increased expression of the gastric epithelial phenotype markers, TFF2 and H/K-ATPase, after treatment with gastric tissue extract (FIG. 14E), demonstrating a similarity in phenotype with bone marrow derived cultured MSCs. However, there was no significant difference in the expression of K19 or other markers that were tested.

Materials and Methods

Mice: All mice studies and breeding were carried out under the approval of IACUC of Columbia University. Ninety-eight mice were used in this study, and 75 of them, including both C57BL/6 mice (8-10 week-old) and chicken beta actin EGFP transgenic mice (8-10 week-old), were purchased from Jackson Laboratories (Bar Harbor, Me.). Twenty-three study mice were chimeric mice derived from blastocyst injection (Table 1).

Isolation and culture of MSCs: Bone marrow cells were collected by flushing femurs and tibias with Hank's balanced salt solution (HBSS) and plated at a density of 10⁶ cells/cm² in murine mesenchymal medium with murine mesenchymal supplements (MesenCult, Stem Cell Technologies, Vancouver, Canada). MSC cultures were derived from 5 WT B6 mice and 5 chicken beta actin EGFP transgenic mice and maintained individually. Non-adherent cells were removed after 24 hrs, and culture media were replaced every 5 days. The cells became confluent within 2-3 weeks at 37° C. in the humid air containing 5% CO2. Adherent cells were detached by 0.25% Trypsin and 0.02% ethylene diaminetetraacetic acid (EDTA) at 37° C. for 2 min and subsequently passaged in the ratio of 1:3 to achieve the required number. The cells of fifth to tenth passage were used for the following protocols.

Colony forming units-fibroblast (CFU-F) assay: MSCs were plated at a density of 5×10⁵ cells/cm² and maintained as described above. At day 14, the medium was removed from the wells, washed twice with Phosphate Buffered Saline (PBS) and fixed/stained with 3% Crystal violet in 100% methanol for 10 minutes at room temperature. Cells were washed with PBS and colonies were counted.

Differentiation Assays: To induce adipocyte differentiation, the subconfluent cells were cultured with MesenCult Stem Cell Medium containing 5.0 μg/mL insulin, 50 μM indomethacin, 1 μM dexamethasone and 0.5 μM 3-Isobutyl-1-methylxanthine (IBMX). After 14 days, these cells were fixed with 4% paraformaldehyde for 15 min, and stained with Oil Red-O.

To induce osteocyte differentiation, the subconfluent cells were cultured with MesenCult Stem Cell Medium containing 1 nM Dexamethasone, 20 mM β-glycerolphosphate, 50 μM L-ascorbic acid 2-phosphate sesquimagnesium salt, and 50 ng/mL L-thyroxine sodium pentahydrate. After 14 days, these cells were fixed with 4% paraformaldehyde for 15 min, and characterization was performed by Alizarin Red staining, which detects calcium deposition.

Induction of expression of gastric phenotype markers in vitro: A total of 5 stomachs from wild type C57BL/6 mice, aged 8-12 weeks, were used for gastric tissue extract, and each experiment required gastric tissue extract from one stomach. The paste from one stomach, made by mortar and pestle, was mixed with 10 mL of MesenCult Stem Cell Medium and incubated at 4° C. for 24 hours. The mixture was centrifuged by 6000 rpm for 20 minutes and the supernatant was obtained and filtered using 0.2 micro m membrane. MSCs were cultured with the medium containing gastric extract for 5 days at 37° C. in the humid air containing 5% CO2.

Establishment of K19GFP vector and stable transfection: The cDNAs encoding mouse K19 promoter sequence and EGFP gene were cloned by PCR from K19-beta Gal vector (Brembeck FH 2001) and pEGFP N1 vector (Clontech, Mountain View, Calif.), respectively, and subcloned into a pcDNA3.1 plasmid (Invitrogen) in which a neomycin selectable marker were encoded. After sequencing, the DNA plasmids were transfected into MSCs with lipofectamine 2000 (Invitrogen) according to standard protocol (Lipofectamine 10 microL/vector DNA 4 microg). Then cells were cultured with MesenCult Stem Cell Medium containing 150 mg/mL of G418. Colonies were picked up 7 days after transfection and cultured. GFP positive clones were selected according to the observation by fluorescence microscopy and the expression of GFP was confirmed by flow-cytometry.

Gastric wall (“submucosal”) injection of MSCs: Sub-confluent state of MSCs were lifted from the culture plates by treatment with 0.25% trypsin/EDTA solution and the cells ware treated with MesenCult Stem Cell Medium to stop the reaction. The cells were once washed with PBS and cell suspension (10,000,000 cells/1 mL PBS) was prepared. Four weeks old wild type C57Bl/6 mice were anesthetized with inhalation of isoflurane and the center of the upper abdomen was opened by about 1 cm incision and the stomach was lifted to outside the abdomen. About 10 microL of the cell suspension was injected into each of several points of gastric wall by using a fine glass needle. The needle was made from glass pipette by using gas burner. The abdominal wall was closed by 5-0 polypropylene surgical suture. Gastric tissue sections were prepared from mice at 2 weeks after injection to detect GFP-positive cells.

Blastocyst injection of MSCs: Blastocyst injections were performed in the Transgenic Core Facility of Columbia University Medical School. Day 3.5 blastocysts were extracted from the uteri of C57Bl/6J pregnant females. Day-3.5 blastocysts were injected with 10 to 15 GFP-labeled MSCs and implanted in uteri of Swiss Webster pseudopregnant females and the pups were euthanized at 8 weeks of age, and histological or polymerase chain reaction (PCR) analyses were conducted on stomachs, and other organs to detect GFP-labeled cells.

Bone Marrow Transplantation and H. felis infection: Bone marrow transplantation and H. felis infection were carried out as previously described [17]. In brief, 35 C57BL/6 WT female recipients received lethal irradiation (950 cGy) from a Cs137 source, after 3 hours, followed by tail vein infusion of donor whole bone marrow cells (5 million cells in 200 microL). Whole bone marrow cells were prepared from chicken beta actin EGFP transgenic donor mice, as mentioned above. Five of the recipients did not receive donor bone marrow cells infusion and served as additional controls. Fifteen of the study mice (age 3 mos) received inoculation of H. felis by oral gavage, while the other 15 recipients remained uninfected. H. felis (ATCC 49179) was used for oral inoculation as described previously reference [17]. The organism was grown for 48 h at 37° C. under microaerobic conditions on 5% lysed horse blood agar. The bacteria were harvested and inoculated (at a titer of 10¹⁰ organisms per ml) into brain heart infusion broth with 30% glycerol added. The bacterial suspension was frozen at −70° C. Prior to use, aliquots were thawed, analyzed for motility, and cultured for evidence of aerobic or anaerobic bacterial contamination. Brain heart infusion broth containing ˜10¹⁰ colony-forming units of H. felis per ml was used as inoculum. The inocula (0.5 ml) were delivered by gastric intubation into each test mouse three times at 2-day intervals by using a sterile oral catheter [59]. After 1 year of infection, mice were euthanized, and both bone marrow and peripheral blood were extracted and used for MSC culture and mRNA detection.

Flow cytometry: Adherent cells were detached by 0.25% Trypsin and 0.02% EDTA at 37° C. for 2 min, washed with blocking buffer (PBS w/1% fetal bovine serum, FBS), and suspended in the same buffer. Then cells were incubated with phycoerythrin (PE)-conjugated anti mouse Sca-1 (eBioscience, San Diego, Calif.), CD45 (BD Phermingen, San Diego, Calif.), ckit (eBioscience), Flk1 (BD Phermingen), or F4/80 (eBioscience) antibody at 1 micro g/1,000,000 cells for 30 minutes at 4° C. PE-conjugated rat IgG2a antibody (Jackson ImmunoResearch, West Grove, Pa.) was served as isotype controls. The cells were analyzed by using BD LSRII (Becton, Dickinson). 4′,6-diamidino-2-phenylindole (DAPI) was added to exclude dead cells.

Cell Proliferation BrdU ELISA: MSC progenies from K19GFP MSC No. 3 GFP(+) and GFP(−), respectively, were plated in 96-well plate at a concentration of 200,000 cells/well and maintained for 24 hrs at 37° C. in the humid air containing 5% CO2. Then, cells were labeled with 10 microM BrdU (Cell proliferation ELISA, BrdU, Colorimetri, Roche, Indianapolis, Ind.) for 2 hrs, fixed and denatured as the manufacturer's suggestion for 30 min at room temperature, and then labeled with detecting antibodies for 90 min. After three times wash with 1× PBS, we added substrate solution for 30 min, followed by 1M H2SO4, and check the OD450.

Quantitative real-time PCR analysis: Total RNA was isolated using Trizol (Invitrogen, Carlsbad, Calif.), as recommended by the manufacturer. First-strand cDNA was synthesized using the SuperScript First-Strand Synthesis System with SuperScript III reverse transcriptase according to the protocols of the manufacturer (Invitrogen). The cDNA generated was used as a template in real-time PCR reactions with the QuantiTect™ SYBR® green PCR kit (QIAGEN, Maryland, Md.) and were run on an ABI Prism 7300 Sequence Detection System (Applied Biosystems, Branchburg, N.J.). Primer sequences are described in Table 2. Each PCR run included a 15-min activation time at 95° C. as required by the instrument. The three-step cycle included denaturing (94° C., 15 seconds), annealing at 55° C. and extension at 72° C. mRNA quantities were analyzed in duplicate, normalized against GAPDH as an internal control gene. Results are expressed as relative gene expression using the delta delta Ct (ddCt) method.

Immunofluorescence staining of the cells: Cells were grown in wells of Lab-Tek 8-chamber culture slides. Fixed with 4% paraformaldehyde in PBS for 15 min in room temperature and digested with pepsin (Abcam Inc., MA) for 10 min in 37° C. After treatment with 5% FBS in PBS for 30 min in room temperature, cells were incubated with Rabbit anti Cytokeratin 19 antibody (Abcam Inc.) in PBS containing 5% FBS at room temperature for 60 min. After three washes in PBS, cells were incubated with Texas Red conjugated goat anti-Rabbit IgG (Jackson ImmunoResearch) at room temperature for 60 min. Cells were counter stained with DAPI, washed with PBS for three times, and the stained cells were mounted using Vectashield (Vector Laboratories, Inc. CA) for microscopy.

PCR for GFP detection: Genomic DNA from mice was extracted using a Genomic DNA isolation kit (Lamda Biotech, St. Louis). Primers used for detection of GFP gene were shown in Table 2. Primers for GAPDH were used to confirm the presence of template DNA in the reactions. The PCR reactions were performed in 50 μL with 50 ng of DNA, each with 10 mM Tris, pH 8.3, 50 mM KCl, 1.5 mM MgCl2, and 200 μM dNTPs each, 0.4 μM each of forward and reverse primer, and 1.25 U Taq DNA polymerase (Roche Diagnostics, Indianapolis, Ind.). The PCR reactions were performed as follows: loading at 95° C. for 2 minutes, denaturation at 95° C. for 30 seconds, annealing at 55° C. for 30 seconds, and elongation at 72° C. for 1 minute for 30 cycles. For positive controls, DNA templates from stomachs of chicken beta actin EGFP transgenic mice were used. A negative control with only water was performed. All of the PCR reactions were analyzed on 1.5% agarose gels.

Tissue processing and immunofluorescence staining: Mice were deeply anesthetized with inhalation of isoflurane and infused through the heart with PBS and then 4% paraformaldehyde. The stomachs were removed, further fixed with 4% paraformaldehyde for 6 hours at 4° C., then equilibrated in 30% sucrose, embedded in OCT, frozen. Four micron sections were prepared by the Research Histology Service at Columbia University Medical Center. Slides were rinsed with PBS, non-specific staining was blocked with 1% FBS in PBS for 1 hour at room temperature, then Rat anti E-cadherin antibody (Zymed, South San Francisco, Calif.) or mouse anti Hydrogen/Potassium ATPase (H/K-ATPase) beta antibody (Affinity BioReagents, Golden, Colo.) diluted in PBS supplemented with 1% FBS (1:100 dilution for E-cadherin, 1:2000 dilution for H/K-ATPase) were applied. Non-transplanted tissues served as additional negative controls. Following overnight incubation at 4° C., slides were washed three times in PBS, and Texas Red conjugated anti Rat IgG antibody (Jackson ImmunoResearch) or Texas Red conjugated anti mouse IgG antibody (Jackson ImmunoResearch) were applied, respectively, with 1% FBS in PBS (1:300 dilution) and incubated for 1 hour at room temperature. Then slides were stained with DAPI, washed with PBS for three times, and mounted using Vectashield (Vector Laboratories) for microscopy.

Immunohistochemistry to detect GFP protein: Immunohistochemical studies were performed with avidin biotin-peroxidase complex kits (Vector Laboratories, Burlingame, Calif.) according to the manufacturer's instructions. For primary antibody, rabbit anti GFP antibody (Invitrogen) diluted in PBS supplemented with 1% FBS (1:100 dilution) was applied. Diaminobenzidine (Vector Laboratories) was used as the chromogen, and slides were counterstained with Mayer's hematoxylin.

TABLE 1
Mice use in this study:

| | |
|---|---|
| MSC donors WT | 5 |
| MSC donors GFP | 5 |
| Gastric injection recipient--GFP MSC | 10 |
| Gastric injection recipient--K19 MSC | 10 |
| Blastocyst injection chimeric pups--GFP MSC | 10 |
| Blastocyst injection chimeric pups--K19 MSC | 13 |
| Gastric lysate | 5 |
| BMT donors | 5 |
| BMT recipients--Hf− | 15 |
| BMT recipients--Hf+ | 15 |
| BMT recipients--control | 5 |
| Total | 98 |

TABLE 2
Sequence of the Primers Used for quantitative and regular RT-PCR

| Gene | qRT-PCR Forward primer | qRT-PCR Reverse primer | Product size |
|---|---|---|---|
| GAPDH | 5′- gac atc aag aag gtg gtg | 5′- ata cca gga aat gag ctt | 174 bp |
| | aag cag -3′ | gac aaa -3′ | |
| | SEQ ID NO: 1 | SEQ ID NO: 2 | |
| Keratin 19 | 5′- gga ccc gga ccc tcc cga | 5′- ggc gca ggc cgt tga tgt | 205 bp |
| | gat t-3′ | cg-3′ | |
| | SEQ ID NO: 3 | SEQ ID NO: 4 | |
| TFF2 | 5′- gca gtg ctt tga tct tgg | 5′- tca ggt tgg aaa agc agc | 185 bp |
| | atg c -3′ | agt t -3′ | |
| | SEQ ID NO: 5 | SEQ ID NO: 6 | |
| IF | 5′- ccc ggt ccc cac ttc agt | 5′- caa taa ggc ccc agg atg | 200 bp |
| | atc t-3′ | tca t-3′ | |
| | SEQ ID NO: 7 | SEQ ID NO: 8 | |
| CgA | 5′- gca gca tcc agt tcc cac | 5′- tcc cca tct tcc tcc tgc | 146 bp |
| | ttc c-3′ | tga g-3′ | |
| | SEQ ID NO: 9 | SEQ ID NO: 10 | |
| H/KATPase- | 5′- gca gac cat tga ccc cta | 5′- agg cca gcc cag gaa ctg | 138 bp |
| beta | cac c-3′ | ttt t-3′ | |
| | SEQ ID NO: 11 | SEQ ID NO: 12 | |
| Mucin5ac | 5′- agg gcc cag tga gca tct | 5′- cat cat cgc agc gca gag | 150 bp |
| | cct a-3′ | tca -3′ | |
| | SEQ ID NO: 13 | SEQ ID NO: 14 | |
| Mucin6 | 5′- ctc acc ttc tac ccc agt | 5′- ggc aac gag tta gag tca | 146 bp |
| | atc a-3′ | cat t -3′ | |
| | SEQ ID NO: 15 | SEQ ID NO: 16 | |
| Nanog | 5′- gca agc ggt ggc aga aaa | 5′- cca agt ctg gct gcc cca | 158 bp |
| | acc -3′ | cat -3′ | |
| | SEQ ID NO: 17 | SEQ ID NO: 18 | |
| | Regular RT-PCR | Regular RT-PCR | |
| Gene | Forward primer | Reverse primer | Product size |
| GAPDH | 5′- gaa gac tgt gga tgg ccc | 5′- gtc cac cac cct gtt gct | 424 bp |
| | ct -3′ | gt -3′ | |
| | SEQ ID NO: 19 | SEQ ID NO: 20 | |
| Nanog | 5′- agg gcc ctg agg agg agg | 5′- tgg ccg ttc cag gac tga | 475 bp |
| | ag -3′ | gc -3′ | |
| | SEQ ID NO: 21 | SEQ ID NO: 22 | |
| Oct3/4 | 5′- gtt ctg cgg agg gat ggc | 5′- aag gcc tcg aag cga cag | 360 bp |
| | ata c -3′ | atg -3′ | |
| | SEQ ID NO: 23 | SEQ ID NO: 24 | |
| GFP | 5′- gag ctg aag ggc atc gac | 5′- gga ctg ggt gct cag gta | 246 bp |
| | ttc aag -3′ | gtg g -3′ | |
| | SEQ ID NO: 25 | SEQ ID NO: 26 | |

### REFERENCES

1. Theise N D, Badve S, Saxena R, et al. Derivation of hepatocytes from bone marrow cells in mice after radiation-induced myeloablation. Hepatology. 2000; 31:235-240.
2. Spencer H, Rampling D, Aurora P, et al. Transbronchial biopsies provide longitudinal evidence for epithelial chimerism in children following sex mismatched lung transplantation. Thorax. 2005; 60:60-62.
3. Suratt B T, Cool C D, Serls A E, et al. Human pulmonary chimerism after hematopoietic stem cell transplantation. Am J Respir Crit Care Med. 2003; 168:318-322.
4. Korbling M, Katz R L, Khanna A, et al. Hepatocytes and epithelial cells of donor origin in recipients of peripheral-blood stem cells. N Engl J Med. 2002; 346:738-746.
5. Okamoto R, Yajima T, Yamazaki M, et al. Damaged epithelia regenerated by bone marrow-derived cells in the human gastrointestinal tract. Nat Med. 2002; 8:1011-1017.
6. Ishikawa F, Yasukawa M, Yoshida S, et al. Human cord blood- and bone marrow-derived CD34+ cells regenerate gastrointestinal epithelial cells. FASEB J. 2004; 18:1958-1960.
7. Matsumoto T, Okamoto R, Yajima T, et al. Increase of bone marrow-derived secretory lineage epithelial cells during regeneration in the human intestine. Gastroenterology. 2005; 128:1851-1867.
8. Spyridonidis A, Schmitt-Graff A, Tomann T, et al. Epithelial tissue chimerism after human hematopoietic cell transplantation is a real phenomenon. Am J Pathol. 2004; 164:1147-1155.
9. Chakraborty A, Lazova R, Davies S, et al. Donor DNA in a renal cell carcinoma metastasis from a bone marrow transplant recipient. Bone Marrow Transplant. 2004; 34:183-186.
   10. Cogle C R, Theise N D, Fu D, et al. Bone marrow contributes to epithelial cancers in mice and humans as developmental mimicry. Stem Cells. 2007; 25:1881-1887.
11. Avital I, Moreira A, Downey R J. The origin of epithelial neoplasms after allogeneic stem cell transplantation. Haematologica. 2006; 91:ELT07; author reply ELT06.
12. Petersen B E, Bowen W C, Patrene K D, et al. Bone marrow as a potential source of hepatic oval cells. Science 1999; 284:1168-1170
13. Krause D S, Theise N D, Collector M I, et al. Multi-organ, multi-lineage engraftment by a single bone marrow-derived stem cell. Cell. 2001; 105:369-377.
14. Lagasse E, Connors H, Al-Dhalimy M, et al. Purified hematopoietic stem cells can differentiate into hepatocytes in vivo. Nat Med 2000; 6:1229-1234
15. Poulsom R, Forbes S J, Hodivala-Dilke K, et al. Bone marrow contributes to renal parenchymal turnover and regeneration. J Pathol. 2001; 195:229-235.
16. Bruscia E M, Ziegler E C, Price J E, et al. Engraftment of donor-derived epithelial cells in multiple organs following bone marrow transplantation into newborn mice. Stem Cells. 2006; 24:2299-2308.
17. Houghton J, Stoicov C, Nomura S, et al. Gastric cancer originating from bone marrow-derived cells. Science. 2004; 306:1568-1571.
18. Herzog E L, Krause D S. Engraftment of marrow-derived epithelial cells: the role of fusion. Proc Am Thorac Soc. 2006; 3:691-695.
19. Friedenstein A J, Chailakhjan R K, Lalykina K S. The development of fibroblast colonies in monolayer cultures of guinea-pig bone marrow and spleen cells. Cell Tissue Kinet. 1970; 3:393-403.
20. Pittenger M F, Mackay A M, Beck S C, et al. Multilineage potential of adult human mesenchymal stem cells. Science. 1999; 284:143-147.
21. Friedenstein A J, Chailakhyan R K, Latsinik N V, et al. Stromal cells responsible for transferring the microenvironment of the hemopoietic tissues. Cloning in vitro and retransplantation in vivo. Transplantation. 1974; 17:331-340.
22. Friedenstein A J, Deriglasova U F, Kulagina N N, et al. Precursors for fibroblasts in different populations of hematopoietic cells as detected by the in vitro colony assay method. Exp Hematol. 1974; 2:83-92.
23. Ferrari G, Cusella-De Angelis G, Coletta M, et al. Muscle regeneration by bone marrow-derived myogenic progenitors. Science. 1998; 279:1528-1530.
24. Cho K J, Trzaska K A, Greco S J, et al. Neurons derived from human mesenchymal stem cells show synaptic transmission and can be induced to produce the neurotransmitter substance P by interleukin-1 alpha. Stem Cells. 2005; 23:383-391.
25. Pittenger M F, Martin B J. Mesenchymal stem cells and their potential as cardiac therapeutics. Circ Res; 2004 Jul. 9; 95(1):9-20. Review.
26. Rojas M, Xu J, Woods C R, et al. Bone marrow-derived mesenchymal stem cells in repair of the injured lung. Am J Respir Cell Mol Biol. 2005; 33:145-152.
27. Mackenzie T C, Shaaban A F, Radu A, et al. Engraftment of bone marrow and fetal liver cells after in utero transplantation in MDX mice. J Pediatr Surg. 2002; 37:1058-1064.
28. Mackenzie T C, Flake A W. Multilineage differentiation of human MSC after in utero transplantation. Cytotherapy. 2001; 3:403-405.
29. Anjos-Afonso F, Bonnet D. Nonhematopoietic/endothelial SSEA-1+ cells define the most primitive progenitors in the adult murine bone marrow mesenchymal compartment. Blood. 2007; 109:1298-1306.
30. Jiang Y, Jahagirdar B N, Reinhardt R L, et al. Pluripotency of mesenchymal stem cells derived from adult marrow. Nature. 2002; 418:41-49.
31. Nadri S, Soleimani M, Kiani J, et al. Multipotent mesenchymal stem cells from adult human eye conjunctiva stromal cells. Differentiation. 2007.
32. D'Ippolito G, Diabira S, Howard G A, et al. Marrow-isolated adult multilineage inducible (MIAMI) cells, a unique population of postnatal young and old human cells with extensive expansion and differentiation potential. J Cell Sci. 2004; 117:2971-2981.
33. Moll R, Franke W W, Schiller D L, et al. The catalog of human cytokeratins: patterns of expression in normal epithelia, tumors and cultured cells. Cell. 1982; 31:11-24.
34. Michel M, Torok N, Godbout M J, et al. Keratin 19 as a biochemical marker of skin stem cells in vivo and in vitro: keratin 19 expressing cells are differentially localized in function of anatomic sites, and their number varies with donor age and culture stage. J Cell Sci. 1996; 109 (Pt 5):1017-1028.
35. Brembeck F H, Moffett J, Wang T C, et al. The keratin 19 promoter is potent for cell-specific targeting of genes in transgenic mice. Gastroenterology. 2001; 120:1720-1728.
36. Calnek D, Quaroni A. Changes in keratin expression during fetal and postnatal development of intestinal epithelial cells. Biochem J. 1992; 285 (Pt 3):939-946.
37. Calnek D, Quaroni A. Differential localization by in situ hybridization of distinct keratin mRNA species during intestinal epithelial cell development and differentiation. Differentiation. 1993; 53:95-104.
38. Quaroni A, Calnek D, Quaroni E, et al. Keratin expression in rat intestinal crypt and villus cells. Analysis with a panel of monoclonal antibodies. J Biol Chem. 1991; 266:11923-11931.
39. Chun-mao H, Su-yi W, Ping-ping L, et al. Human bone marrow-derived mesenchymal stem cells differentiate into epidermal-like cells in vitro. Differentiation. 2007; 75:292-298.
40. Digirolamo C M, Stokes D, Colter D, et al. Propagation and senescence of human marrow stromal cells in culture: a simple colony-forming assay identifies samples with the greatest potential to propagate and differentiate. Br J Haematol. 1999; 107:275-281.
41. Colter D C, Class R, DiGirolamo C M, et al. Rapid expansion of recycling stem cells in cultures of plastic-adherent cells from human bone marrow. Proc Natl Acad Sci USA. 2000; 97:3213-3218.
42. Javazon E H, Colter D C, Schwarz E J, et al. Rat marrow stromal cells are more sensitive to plating density and expand more rapidly from single-cell-derived colonies than human marrow stromal cells. Stem Cells. 2001; 19:219-225.
43. Phinney D G, Kopen G, Isaacson R L, et al. Plastic adherent stromal cells from the bone marrow of commonly used strains of inbred mice: variations in yield, growth, and differentiation. J Cell Biochem. 1999; 72:570-585.
44. Kadiyala S, Young R G, Thiede M A, et al. Culture expanded canine mesenchymal stem cells possess osteochondrogenic potential in vivo and in vitro. Cell Transplant. 1997; 6:125-134.
45. Martin D R, Cox N R, Hathcock T L, et al. Isolation and characterization of multipotential mesenchymal stem cells from feline bone marrow. Exp Hematol. 2002; 30:879-886.
46. Javazon E H, Beggs K J, Flake A W. Mesenchymal stem cells: paradoxes of passaging. Exp Hematol. 2004; 32:414-425.
47. Owen M, Friedenstein A J. Stromal stem cells: marrow-derived osteogenic precursors. Ciba Found Symp. 1988; 136:42-60.
48. Houghton J, Wang T C. Role of bone marrow-derived cells (BMDCs) in gastric adenocarcinoma, Springer Science Press, 2007
49. Yoon Y S, Wecker A, Heyd L, et al. Clonally expanded novel multipotent stem cells from human bone marrow regenerate myocardium after myocardial infarction. J Clin Invest. 2005; 115:326-338.
50. Reyes M, Lund T, Lenvik T, et al. Purification and ex vivo expansion of postnatal human marrow mesodermal progenitor cells. Blood. 2001; 98:2615-2625.
51. Li H, Fan X, Kovi R C, Jo Y, et al. Spontaneous Expression of Embryonic Factors and p53 Point Mutations in Aged Mesenchymal Stem Cells: A Model of Age-Related Tumorigenesis In Mice. Cancer Res. 2007; 67:10889-10898.
52. Kuznetsov S A, Mankani M H, Gronthos S, et al. Circulating skeletal stem cells. J Cell Biol. 2001; 153:1133-1140.
53. Metcalf D. Formation in agar of fibroblast-like colonies by cells from the mouse pleural cavity and other sources. J Cell Physiol. 1972; 80:409-419.
54. Piersma A H, Ploemacher R E, Brockbank K G, et al. Migration of fibroblastoid stromal cells in murine blood. Cell Tissue Kinet. 1985; 18:589-595.
55. Rochefort G Y, Delorme B, Lopez A, et al. Multipotential mesenchymal stem cells are mobilized into peripheral blood by hypoxia. Stem Cells. 2006; 24:2202-2208.
56. Kassis I, Zangi L, Rivkin R, et al. Isolation of mesenchymal stem cells from G-CSF-mobilized human peripheral blood using fibrin microbeads. Bone Marrow Transplant. 2006; 37:967-976.
57. Swenson E S, Price J G, Brazelton T et al. Limitations of green fluorescent protein as a cell lineage marker. Stem Cells. 2007; 25:2593-2600.
58. Okabe M, Ikawa M, Kominami K et al. ‘Green mice’ as a source of ubiquitous green cells. FEBS Lett. 1997; 407:313-319.
59. Fox J G, Blanco M, Murphy J C et al. Local and systemic immune responses in murine Helicobacter felis active chronic gastritis. Infect Immun. 1993; 61:2309-2315.

Ai W, Zheng H, Yang X, Liu Y, Wang T C. Tip60 functions as a potential corepressor of KLF4 in regulation of HDC promoter activity. Nucleic Acids Res. 2007; 35(18):6137-49. Epub 2007 Sep. 7.

Ai W, Liu Y, Langlois M, Wang T C. Kruppel-like factor 4 (KLF4) represses histidine decarboxylase gene expression through an upstream Sp1 site and downstream gastrin responsive elements. J Biol Chem. 2004 Mar. 5; 279(10):8684-93. Epub 2003 Dec. 10. Erratum in: J Biol Chem. 2004 Jun. 25; 279(26):27830.

Anjos-Afonso F, Bonnet D. Nonhematopoietic/endothelial SSEA-1+ cells define the most primitive progenitors in the adult murine bone marrow mesenchymal compartment. Blood. 2007; 109:1298-1306.

Aoi T, Yae K, Nakagawa M, Ichisaka T, Okita K, Takahashi K, Chiba T, Yamanaka S. Generation of Pluripotent Stem Cells from Adult Mouse Liver and Stomach Cells. Science. 2008 Feb. 14. [Epub ahead of print]

Brambrink T, Foreman R, Welstead G G, Lengner C J, Wernig M, Suh H, Jaenisch R. Sequential expression of pluripotency markers during direct reprogramming of mouse somatic cells. Cell Stem Cell. 2008 Feb. 7; 2(2):151-9.

Chun-mao H, Su-yi W, Ping-ping L, et al. Human bone marrow-derived mesenchymal stem cells differentiate into epidermal-like cells in vitro. Differentiation. 2007; 75:292-298.

D'Ippolito G, Diabira S, Howard G A, et al. Marrow-isolated adult multilineage inducible (MIAMI) cells, a unique population of postnatal young and old human cells with extensive expansion and differentiation potential. J Cell Sci. 2004; 117:2971-2981.

Ferrari G, Cusella-De Angelis G, Coletta M, et al. Muscle regeneration by bone marrow-derived myogenic progenitors. Science. 1998; 279:1528-1530.

Friedenstein A J, Chailakhyan R K, Latsinik N V, et al. Stromal cells responsible for transferring the microenvironment of the hemopoietic tissues. Cloning in vitro and retransplantation in vivo. Transplantation. 1974; 17:331-340.

Hanna J, Wernig M, Markoulaki S, Sun C W, Meissner A, Cassady J P, Beard C, Brambrink T, Wu L C, Townes T M, Jaenisch R. Treatment of sickle cell anemia mouse model with iPS cells generated from autologous skin. Science. 2007 Dec. 21; 318(5858):1920-3. Epub 2007 Dec. 6.

Hanna J, Markoulaki S, Schorderet P, Carey B W, Beard C, Wernig M, Creyghton M P, Steine E J, Cassady J P, Foreman R, Lengner C J, Dausman J A, Jaenisch R. Direct reprogramming of terminally differentiated mature B lymphocytes to pluripotency. Cell. 2008 Apr. 18; 133(2):250-64.

Houghton J, Stoicov C, Nomura S, et al. Gastric cancer originating from bone marrow-derived cells. Science. 2004; 306:1568-1571.

Huangfu D, Maehr R, Guo W, Eijkelenboom A, Snitow M, Chen A E, Melton D A. Induction of pluripotent stem cells by defined factors is greatly improved by small-molecule compounds. Nat Biotechnol. 2008 Jun. 22. [Epub ahead of print]

Hyun I, Hochedlinger K, Jaenisch R, Yamanaka S. New advances in iPS cell research do not obviate the need for human embryonic stem cells. Cell Stem Cell. 2007 Oct. 11; 1(4):367-8. No abstract available.

Jiang Le, Gonda T A, Gamble M, Seshan V, Tu S, Twaddell W S, Hegyi P, Varro A, Wang T C, Tycko B. Global hypomethylatoin of genomic DNA in cancer-associated myofibroblasts. Cancer Research 2008 (in press).

Jiang Y, Jahagirdar B N, Reinhardt R L, et al. Pluripotency of mesenchymal stem cells derived from adult marrow. Nature. 2002; 418:41-49.

Krause D S, Theise N D, Collector M I, et al. Multi-organ, multi-lineage engraftment by a single bone marrow-derived stem cell. Cell. 2001; 105:369-377.

Mackenzie T C, Flake A W. Multilineage differentiation of human MSC after in utero transplantation. Cytotherapy. 2001; 3:403-405.

Maherali N, Sridharan R, Xie W, Utikal J, Eminli S, Arnold K, Stadtfeld M, Yachechko R, Tchieu J, Jaenisch R, Plath K, Hochedlinger K. Directly reprogrammed fibroblasts show global epigenetic remodeling and widespread tissue contribution. Cell Stem Cell. 2007 Jun. 7; 1(1):55-70.

Meissner A, Wernig M, Jaenisch R. Direct reprogramming of genetically unmodified fibroblasts into pluripotent stem cells. Nat Biotechnol. 2007 October; 25(10):1177-81. Epub 2007 Aug. 27.

Moll R, Franke W W, Schiller D L, et al. The catalog of human cytokeratins: patterns of expression in normal epithelia, tumors and cultured cells. Cell. 1982; 31:11-24.

Nadri S, Soleimani M, Kiani J, et al. Multipotent mesenchymal stem cells from adult human eye conjunctiva stromal cells. Differentiation. 2007.

Nakagawa M, Koyanagi M, Tanabe K, Takahashi K, Ichisaka T, Aoi T, Okita K, Mochiduki Y, Takizawa N, Yamanaka S. Generation of induced pluripotent stem cells without Myc from mouse and human fibroblasts. Nat Biotechnol. 2008 January; 26(1):101-6. Epub 2007 Nov. 30.

Okamoto R, Yajima T, Yamazaki M, et al. Damaged epithelia regenerated by bone marrow-derived cells in the human gastrointestinal tract. Nat Med. 2002; 8:1011-1017.

Okita K, Ichisaka T, Yamanaka S. Generation of germline-competent induced pluripotent stem cells. Nature. 2007 Jul. 19; 448(7151):313-7.

Park I H, Zhao R, West J A, Yabuuchi A, Huo H, Ince T A, Lerou P H, Lensch M W, Daley G Q. Reprogramming of human somatic cells to pluripotency with defined factors. Nature. 2008 Jan. 10; 451(7175):141-6. Epub 2007 Dec. 23.

Pittenger M F, Mackay A M, Beck S C, et al. Multilineage potential of adult human mesenchymal stem cells. Science. 1999; 284:143-147.

Stadtfeld M, Brennand K, Hochedlinger K. Reprogramming of Pancreatic beta Cells into Induced Pluripotent Stem Cells. Curr Biol. 2008 May 21. [Epub ahead of print]

Suratt B T, Cool C D, Serls A E, et al. Human pulmonary chimerism after hematopoietic stem cell transplantation. Am J Respir Crit Care Med. 2003; 168:318-322.

Takahashi K, Yamanaka S. Induction of pluripotent stem cells from mouse embryonic and adult fibroblast cultures by defined factors. Cell. 2006 Aug. 25; 126(4):663-76. Epub 2006 Aug. 10.

Takahashi K, Tanabe K, Ohnuki M, Narita M, Ichisaka T, Tomoda K, Yamanaka S. Induction of pluripotent stem cells from adult human fibroblasts by defined factors. Cell. 2007a Nov. 30; 131(5):861-72.

Takahashi K, Okita K, Nakagawa M, Yamanaka S. Induction of pluripotent stem cells from fibroblast cultures. Nat Protoc. 2007b; 2(12):3081-9.

Theise N D, Badve S, Saxena R, et al. Derivation of hepatocytes from bone marrow cells in mice after radiation-induced myeloablation. Hepatology. 2000; 31:235-240.

Wernig M, Meissner A, Foreman R, Brambrink T, Ku M, Hochedlinger K, Bernstein B E, Jaenisch R. In vitro reprogramming of fibroblasts into a pluripotent ES-cell-like state. Nature. 2007 Jul. 19; 448(7151):318-24. Epub 2007 Jun. 6.

Wernig M, Zhao J P, Pruszak J, Hedlund E, Fu D, Soldner F, Broccoli V, Constantine-Paton M, Isacson O, Jaenisch R. Neurons derived from reprogrammed fibroblasts functionally integrate into the fetal brain and improve symptoms of rats with Parkinson's disease. Proc Natl Acad Sci USA. 2008 Apr. 15; 105(15):5856-61. Epub 2008 Apr. 7.

Yamanaka S. Strategies and new developments in the generation of patient-specific pluripotent stem cells. Cell Stem Cell. 2007 Jun. 7; 1(1):39-49.

Yu J, Vodyanik M A, Smuga-Otto K, Antosiewicz-Bourget J, Frane J L, Tian S, Nie J, Jonsdottir G A, Ruotti V, Stewart R, Slukvin I I, Thomson J A. Induced pluripotent stem cell lines derived from human somatic cells. Science. 2007 Dec. 21; 318(5858):1917-20. Epub 2007 Nov. 20.

1. (canceled)

2. A method for making iPS cells from mesenchymal stem cells comprising: expressing Oct3/4, or expressing Oct3/4 and Sox2 in isolated MSCs, and culturing the MSCs under appropriate conditions, thereby converting, at least a subset of, the population of MSCs into iPS cells, or converted cells with higher level of potency compared to the isolated MSCs, or converted cells which have at least some characteristics of ES cells, such as but not limited to morphology, growth/doubling time, gene expression profile, potency potential, or any combination thereof.

3. (canceled)

4. (canceled)

5. The method of claim 2, optionally comprising a step of identifying MSCs which have increased level of Oct3/4 expression, Sox2 expression, or both, wherein the optional step is carried out after step (a).

6. (canceled)

7. (canceled)

8. The method of claim 2, wherein the method does not comprise a step of transfecting, contacting or exposing MSCs to cMyc, KLF4, Sox2, Nanog, Lin28, or any combination thereof.

9. The method of claim 2, wherein the method does not comprise a step of transfecting, contacting or exposing MSCs to cMyc, KLF4, or the combination thereof.

10. (canceled)

11. (canceled)

12. The method of claim 2, wherein the MSC are human MSCs.

13. The method of claim 2, wherein the isolated MSCs comprise subpopulations of MSCs which express any of K19, KLF4, c-Myc, Sox2, Nanog, or any combination thereof.

14. The method of claim 13, wherein the isolated MSCs do not express detectable levels of Oct3/4.

15. The method of claim 2, wherein the isolated MSCs comprise a subpopulation of MSCs which are CD44+, SSEA1+ and are Lin(−), CD45(−).

16. The method of claim 2, wherein the isolated MSCs comprise a subpopulation of MSCs which are CD44+, and are Lin(−), CD45(−).

17. The method of claim 2, wherein the isolated MSCs comprise subpopulations of MSCs which express higher levels of any of K19, KLF4, or any combination thereof compared to the rest of the MSCs in the population.

18. (canceled)

19. (canceled)

20. A method for making subject specific iPS cells comprising:
a) isolating MSCs from a subject;
b) exposing the isolated MSCs to Oct3/4, or Oct3/4 and Sox2; and
c) culturing the MSCs of step (b) under appropriate conditions, thereby converting (at least a subset of) the MSCs into subject specific iPS cells.

21. An iPS cell obtained by the method of claim 2.

22. A converted cell obtained by the method of claim 2-4.

23. The method of claim 2, wherein the MSCs are obtained from a post-natal individual.

24. The method of claim 2, wherein the MSCs are obtained from the bone marrow of a subject.

25. (canceled)

26. A population of isolated MSCs which express K19, KLF4, c-Myc, Sox2, Nanog, or any combination thereof.

27. The population of claim 26, wherein the levels K19, KLF4, or any combination thereof are increased compared to a general population of isolated MSCs.

28. The population of claim 26, wherein the isolated MSCs do not express detectable levels of Oct3/4.

29. A sub-population of MSCs which are CD44+, and are Lin(−) and CD45(−).

30. The sub-population of MSCs of claim 29, which are SSEA1+.

31. (canceled)
